(19) United States
(12) Patent Application Publication
(10) Pub. No.: US 2026/0216201 A1
(43) Pub. Date: Jul. 30, 2026

(54) PHARMACEUTICAL COMPOSITIONS COMPRISING MELOXICAM

(71) Applicant: AXSOME THERAPEUTICS, INC., New York, NY (US)

(72) Inventor: Herriot Tabuteau, New York, NY (US)

(21) Appl. No.: 19/306,984

(22) Filed: Aug. 21, 2025

Related U.S. Application Data
(60) Provisional application No. 63/751,735, filed on Jan. 30, 2025.
(60) Provisional application No. 63/751,707, filed on Jan. 30, 2025.

(51) Int. Cl. A61K 31/5415; A61K 31/4196; A61P 25/06

(52) U.S. Cl. CPC A61K 31/5415; A61K 31/4196; A61P 25/06

(57) ABSTRACT

Disclosed herein are compositions comprising an NSAID such as meloxicam and/or rizatriptan in combination with a cyclodextrin and/or a bicarbonate. These compositions may be orally administered, for example, to improve the bioavailability or pharmacokinetics of meloxicam and/or rizatriptan for the treatment of pain such as migraine, arthritis, and other conditions. Also disclosed herein are methods of treating pain, such as migraine, comprising administering meloxicam and rizatriptan to a human being suffering from pain, such as migraine. For migraine, these methods may be particularly useful when the meloxicam and rizatriptan are administered while the human being is suffering from an acute attack of migraine pain or migraine aura.

### CROSS-REFERENCE TO RELATED APPLICATIONS

This application claims the benefit of U.S. provisional patent application Nos. 63/751,735, filed Jan. 30, 2025, and 63/751,707, filed Jan. 30, 2025; which are incorporated by reference herein in their entirety.

### SUMMARY

Some embodiments include a method of treating migraine, comprising administering a tablet containing 20 mg of meloxicam, or a molar equivalent amount of a salt of meloxicam, and 10 mg of rizatriptan, or a molar equivalent amount of a salt of rizatriptan, to a human patient at the onset of a migraine, wherein the human patient is not experiencing hemiplegic migraine or basilar migraine. The combination of meloxicam and rizatriptan may not be suitable for use in the management of hemiplegic or basilar migraine.

Some embodiments include a method of treating migraine, comprising: administering a tablet containing 20 mg of meloxicam, or a molar equivalent amount of a salt of meloxicam, and 10 mg of rizatriptan, or a molar equivalent amount of a salt of rizatriptan, to a human patient with a clear diagnosis of migraine at the onset of a migraine attack, wherein if the human patient has no response after the tablet is administered, the diagnosis of migraine is reconsidered, and if the clear diagnosis of migraine is confirmed, administering the tablet during a subsequent migraine attack.

In some embodiments, the combination of meloxicam and rizatriptan should be used where a clear diagnosis of migraine has been established. In some embodiments, if a patient has no response for the first migraine attack treated with the combination of meloxicam and rizatriptan, the diagnosis of migraine should be reconsidered before the combination of meloxicam and rizatriptan is administered to treat any subsequent attacks.

### BRIEF DESCRIPTION OF THE DRAWINGS

FIG. 1 presents the percentage of patients achieving migraine pain freedom within 2 hours following treatment in Study 1.

FIG. 2 presents the percentage of patients achieving MBS freedom within 2 hours following treatment Study 1.

FIG. 3 presents the percentage of patients achieving migraine pain freedom within 2 hours following treatment in Study 2.

FIG. 4 presents the percentage of patients achieving MBS freedom within 2 hours following treatment Study 2.

### DETAILED DESCRIPTION

This disclosure relates to a combination of meloxicam (an NSAID) and rizatriptan (a serotonin (5-HT) 1B/1D receptor agonist or triptan) for the acute treatment of migraine with or without aura in adults. For convenience, dosage form comprising a combination of meloxicam and rizatriptan as described herein is referred to as the “subject dosage form.” In some embodiments, the subject dosage form (e.g., one tablet) is administered by mouth at the onset of a migraine.

Meloxicam has the molecular formula C₁₄H₁₃N₃O₄S₂ and is chemically designated as 4-hydroxy-2-methyl-N-(5 methyl-2-thiazolyl)-2H-1,2-benzothiazine-3-carboxamide-1,1-dioxide. It has a molecular weight of 351.4 g/mole.

The structural formula is:

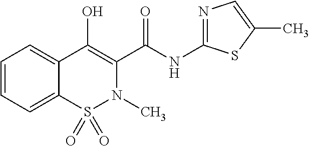 [SMILES: Cc1cnc(NC(=O)C2=C(O)c3ccccc3S(=O)(=O)N2C)s1]

Meloxicam is a pastel yellow solid, practically insoluble in water, with higher solubility observed in strong acids and bases. It is very slightly soluble in methanol. Meloxicam has an apparent partition coefficient (log P)=0.1 in n-octanol/buffer pH 7.4. Meloxicam has pKa values of 1.1 and 4.2.

Rizatriptan benzoate has the molecular formula C₁₅H₁₉N₅·C₇H₆O₂ and is chemically designated as N,N dimethyl-5-(1H-1,2,4-triazol-1-ylmethyl)-1H indole-3 ethanamine monobenzoate. The molecular weight of the free base rizatriptan is 269.4 g/mole. The structural formula is:

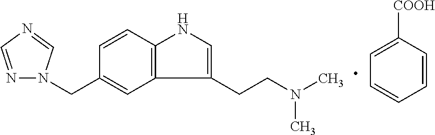 [SMILES: CN(C)CCc1c[nH]c2ccc(Cn3cncn3)cc12 ; O=C(O)c1ccccc1]

Rizatriptan benzoate is a white to off-white, crystalline solid that is soluble in water at about 42 mg per mL (expressed as free base) at 25° C.

Unless otherwise indicated, any reference to a compound herein, such as rizatriptan or meloxicam, by structure, name, or any other means, includes pharmaceutically acceptable salts; alternate solid forms, such as polymorphs, crystals, solvates, hydrates, etc.; tautomers; deuterium-modified compounds, such as deuterium modified dextromethorphan; or any chemical species that may rapidly convert to a compound described herein under conditions in which the compounds are used as described herein.

A dosage form, such as a tablet, may contain, for example, about 18-22 mg, such as 20 mg of meloxicam free base, or a molar equivalent amount of a salt form of meloxicam, and about 9-11 mg, such as about 10 mg of rizatriptan free base, or a molar equivalent amount of a salt form of rizatriptan (e.g., about 14-15 mg, such as about 14.5 mg, of rizatriptan benzoate, or a molar equivalent amount of another salt form of rizatriptan).

A dosage form, such as a tablet, may also contain: colloidal silicon dioxide, crospovidone, magnesium stearate, microcrystalline cellulose, partially hydrolyzed polyvinyl alcohol, polyethylene glycol, povidone, pregelatinized starch, sodium bicarbonate, sulfobutyl-ether-R-cyclodextrin sodium, talc, and/or titanium dioxide.

In some embodiments, the dosage form, such as the tablet, contains colloidal silicon dioxide.

In some embodiments, the dosage form, such as the tablet, contains crospovidone.

In some embodiments, the dosage form, such as the tablet, contains magnesium stearate.

In some embodiments, the dosage form, such as the tablet, contains microcrystalline cellulose.

In some embodiments, the dosage form, such as the tablet, contains partially hydrolyzed polyvinyl alcohol.

In some embodiments, the dosage form, such as the tablet, contains polyethylene glycol.

In some embodiments, the dosage form, such as the tablet, contains povidone.

In some embodiments, the dosage form, such as the tablet, contains pregelatinized starch.

In some embodiments, the dosage form, such as the tablet, contains sodium bicarbonate.

In some embodiments, the dosage form, such as the tablet, contains sulfobutyl-ether-R-cyclodextrin sodium.

In some embodiments, the dosage form, such as the tablet, contains talc.

In some embodiments, the dosage form, such as the tablet, contains titanium dioxide.

In some embodiments, the human patient is screened for a history of asthma, urticaria, or other allergic-type reactions after taking aspirin or other NSAIDs. In some embodiments, the human patient does not have, or is selected for not having a history of asthma, urticaria, or other allergic-type reactions after taking aspirin or other NSAIDs. History of asthma, urticaria, or other allergic-type reactions after taking aspirin or other NSAIDs. Severe, sometimes fatal anaphylactic-like reactions to NSAIDs have been reported in such patients.

In some embodiments, the human patient is screened for history of ischemic heart disease or coronary artery vasospasm. In some embodiments, the human patient does not have, or is selected for not having a history of ischemic heart disease or coronary artery vasospasm.

In some embodiments, the human patient is screened for a history of stroke or transient ischemic attack. In some embodiments, the human patient does not have, or is selected for not having a history of stroke or transient ischemic attack.

In some embodiments, the human patient is screened for a peripheral vascular disease.

In some embodiments, the human patient does not have, or is selected for not having a peripheral vascular disease.

In some embodiments, the human patient is screened for an ischemic bowel disease. In some embodiments, the human patient does not have, or is selected for not having a ischemic bowel disease.

In some embodiments, the human patient is screened for uncontrolled hypertension. In some embodiments, the human patient does not have, or is selected for not having a uncontrolled hypertension.

In some embodiments, the human patient is screened for moderate to severe renal insufficiency. In some embodiments, the human patient does not have, or is selected for not having a moderate to severe renal insufficiency.

In some embodiments, the human patient is screened for recent (within 24 hours) use of an ergotamine-containing medication. In some embodiments, the human patient does not have, or is selected for not having a recent (within 24 hours) use of an ergotamine-containing medication.

In some embodiments, the human patient is screened for use of a MAO-A inhibitor in the past 2 weeks. In some embodiments, the human patient does not have, or is selected for not having a used an MAO-A inhibitor in the past 2 weeks.

In some embodiments, the human patient is screened for hemiplegic or basilar migraine. In some embodiments, the human patient does not have, or is selected for not having a hemiplegic or basilar migraine.

In some embodiments, a subject combination is not administered in the setting of CABG surgery.

In some embodiments, if the patient has multiple cardiovascular risk factors, a cardiac evaluation is performed on the patient. For example, the human patient may be evaluated for myocardial ischemia, myocardial infarction, and/or Prinzmetal's angina.

In some embodiments, the human patient is screened for cluster headaches. In some embodiments, the human patient does not have, or is selected for not having cluster headaches.

In some embodiments, the human patient is screened for tension headaches. In some embodiments, the human patient does not have, or is selected for not having tension headaches.

In some embodiments, the human patient is screened for more than eight migraine headaches during either of the past two months. In some embodiments, the human patient does not have, or is selected for not having more than eight migraine headaches during either of the past two months.

In some embodiments, the human patient is screened for chronic daily headaches, or 15 or more non-migraine headache days/month for the past three months. In some embodiments, the human patient does not have, or is selected for not having chronic daily headaches, or 15 or more non-migraine headache days/month for the past three months.

In some embodiments, the human patient is screened for hemiplegic or basilar migraine. In some embodiments, the human patient does not have, or is selected for not having a hemiplegic or basilar migraine.

In some embodiments, the human patient is screened for hemiplegic or basilar migraine. In some embodiments, the human patient does not have, or is selected for not having a hemiplegic or basilar migraine.

In some embodiments the human patient is, or is selected for being, 18 to 65 years of age inclusive.

In some embodiments the human patient has, or is selected for having, an established diagnosis of migraine (history indicating the presence of migraine for at least 1 year) with or without aura as defined by the International Classification of Headache Disorders 3rd edition (ICHD-3) criteria.

In some embodiments the human patient has, or is selected for having, a diagnosis of migraine attacks with or without aura, presenting before age 50.

In some embodiments the human patient has, or is selected for having, had a history, on average, of 2 to 8 migraine attacks per month over the past 3 months.

In some embodiments the human patient has, or is selected for having, at least 1, and no more than 8, migraine attacks in each of the past three months.

In some embodiments the human patient has, or is selected for having had a history of usual migraine duration of >3 hours untreated (by history) for the past 3 months.

In some embodiments the human patient has, or is selected for having, had a body weight ≥45 kg and a BMI ≤40 kg/m².

In some embodiments the human patient has not, or is selected for not having, received a selective serotonin reuptake inhibitor (SSRI) or serotonin norepinephrine reuptake inhibitor (SNRI); if the human patient is receiving a selective serotonin reuptake inhibitor (SSRI) or serotonin norepinephrine reuptake inhibitor (SNRI), the dose had been stable for at least 8 weeks prior to treatment.

In some embodiments, if female, the human patient is, or is selected for being, either not of childbearing potential (defined as postmenopausal for at least 1 year or surgically sterile [bilateral tubal ligation, bilateral oophorectomy, or hysterectomy]) or nonlactating and nonpregnant (had negative pregnancy test results at screening and baseline), did not plan to get pregnant during the study or for at least 1 month after, and was using a reliable method of contraception, before study drug administration and for the duration of the study. Reliable methods of contraception included hormonal, double-barrier methods (e.g., condom and diaphragm, condom and foam, condom and sponge, each with spermicidal jellies or cream), intrauterine devices, vasectomized partners (>6 months prior to randomization), and abstinence.

In some embodiments the human patient is, or is selected for being, willing and able to complete a Headache Diary.

In some embodiments, the human patient has not, or is selected for not having, a known history of allergic reaction, hypersensitivity, or clinically significant intolerance to rizatriptan or another triptan, acetaminophen, aspirin, or any NSAIDs, including meloxicam; history of NSAID-induced bronchospasm (participants with the triad of asthma, nasal polyps, and chronic rhinitis were at greater risk for bronchospasm and were to be considered carefully); or hypersensitivity, allergy, or significant reaction to any ingredients of the study drug.

In some embodiments, the human patient has not, or is selected for not having, experienced >8 migraine attacks monthly during either of the past 2 months.

In some embodiments, the human patient was not, or is selected for not suffering from cluster headaches (every day or every other day), or other types of migraine.

In some embodiments, the human patient has not, or is selected for not having experienced chronic daily headache (≥15 days per month of non-migraine headaches during each of the prior 3 months).

In some embodiments, the human patient does not, or is selected for not having a history of brain stem aura, ophthalmoplegic or hemiplegic migraine headache, or any potentially serious neurological condition that was associated with headache.

In some embodiments, the human patient has not, or is selected for not having confirmed or suspected cardiovascular or cerebrovascular disease.

In some embodiments, the human patient has not, or is selected for not having history, symptoms, or significant risk factors for ischemic heart (e.g., silent ischemia, angina, myocardial infarction); coronary artery vasospasm; arrhythmia (e.g., atrial fibrillation or flutter, frequent premature ventricular contractions, atrioventricular block); clinically significant findings on ECG; cardiac accessory conduction pathway disorder (e.g., Wolff-Parkinson-White syndrome); or other cardiovascular disease.

In some embodiments, the human patient has not, or is selected for not having history of stroke, transient ischemic attack, or other cerebrovascular syndrome; peripheral vascular disease; or ischemic bowel disease.

In some embodiments, the human patient has not, or is selected for not having uncontrolled hypertension (diastolic blood pressure >95 mm Hg or systolic blood pressure >160 mm Hg).

In some embodiments, the human patient is female, and has not, or is selected for not having was taking estrogenic contraceptives and, in addition, smoked and had experienced migraine attack with aura.

In some embodiments, the human patient has not, or is selected for not having a concurrent medical condition(s) that required the chronic (daily or near daily) use of analgesics, narcotic analgesics, steroidal or nonsteroidal anti-inflammatory agents, tranquilizers, sedatives, hypnotics, antipsychotics, or nitrates or their use for prevention of migraine attacks.

In some embodiments, the human patient has not, or is selected for not having clinically significant abnormalities indicated from the medical history, physical examination, clinical chemistry, hematology, or urine drug screen.

In some embodiments, the human patient has not, or is selected for not having a diagnosis or suspicion of drug-induced or chronic daily headaches within 1 year.

In some embodiments, the human patient has not, or is selected for not having used monoamine oxidase (MAO) inhibitor, lithium, methylergonovine, or cimetidine in the 2 weeks before randomization.

In some embodiments, the human patient has not, or is selected for not having a history or current diagnosis of any clinically significant cardiac, pulmonary, neurological, immunological, hematological, gastrointestinal (GI), hepatic, renal, or endocrine disease.

In some embodiments, the human patient has not, or is selected for not having a history or current diagnosis of schizophrenia or another significant psychiatric disorder.

In some embodiments, the human patient has not, or is selected for not having receiving systemic chemotherapy, had an active malignancy of any type, or had been diagnosed with cancer within the previous 5 years (excluding squamous or basal cell carcinoma of the skin).

In some embodiments, the human patient has not, or is selected for not having a known or suspected history of alcoholism or drug abuse or misuse within the past 1 year.

In some embodiments, the human patient has not, or is selected for not currently having, or within 1 year, any clinically significant GI disorder, including peptic or gastric ulcers or GI bleeding.

In some embodiments, the human patient has not, or is selected for not having a medical or surgical condition of the GI system (including motility dysfunction) or renal system that might significantly alter the absorption, distribution, or excretion of any drug substance.

In some embodiments, the human patient being treated with the subject dosage form is not treated with concurrent analgesic or NSAID.

In some embodiments, the human patient has not, or is selected for not currently receiving or expecting to use anticoagulants (e.g., heparin, warfarin, nutritional supplements having anticoagulant properties) or having bleeding problems, coagulation abnormalities, active blood dyscrasia, hemorrhagic disease, anemia, porphyria, phenylketonuria, bone marrow suppression, or immunosuppression.

In some embodiments, the human patient has not, or is selected for not currently receiving propranolol or had received propranolol within the past 2 weeks.

In some embodiments, the human patient has not, or is selected for not having been treated with agents that could have affected the analgesic response (such as central alpha adrenergic agents [clonidine and tizanidine]) within the past 2 weeks.

A combination of meloxicam and rizatriptan should not be given to patients with ischemic or vasospastic coronary artery disease. There have been rare reports of serious cardiac adverse reactions, including acute myocardial infarction, occurring within a few hours following administration of rizatriptan. Some of these reactions occurred in patients without known coronary artery disease (CAD). 5-HT1 agonists, including a subject combination may cause coronary artery vasospasm (Prinzmetal's Angina), even in patients without a history of CAD.

In some embodiments, triptan-naïve patients who have multiple cardiovascular risk factors (e.g., increased age, diabetes, hypertension, smoking, obesity, strong family history of CAD) have a cardiovascular evaluation prior to receiving a subject combination. In some embodiments, if there is evidence of CAD or coronary artery vasospasm, a subject combination is not administered. In some embodiments, for a human patient who has a negative cardiovascular evaluation, a treatment method comprises administering a first dose of a subject combination in a medically-supervised setting, and performing an electrocardiogram (ECG) on the human patient immediately following administration of a subject combination. In some embodiments, a treatment method may comprise periodic cardiovascular evaluation wherein the human patient is an intermittent long-term users of a subject combination, and the human patient has cardiovascular risk factors.

In some embodiments, the use of a subject combination in patients with a recent myocardial infarction (MI) is avoided unless the benefits are expected to outweigh the risk of recurrent CV thrombotic events. In some embodiments, if a subject combination is used in a human patient with a recent MI, the human patient is monitored for signs of cardiac ischemia.

In some embodiments, the human patient is screened for ischemic coronary artery disease (angina pectoris, history of myocardial infarction, or documented silent ischemia), or other significant underlying cardiovascular disease. In some embodiments, the human patient does not have, or is selected for not having ischemic coronary artery disease (angina pectoris, history of myocardial infarction, or documented silent ischemia), or other significant underlying cardiovascular disease.

In some embodiments, the human patient is screened for coronary artery vasospasm including Prinzmetal's angina. In some embodiments, the human patient does not have, or is selected for not having coronary artery vasospasm including Prinzmetal's angina.

In some embodiments, the human patient is screened for a history of stroke or transient ischemic attack (TIA). In some embodiments, the human patient does not have, or is selected for not having a history of stroke or transient ischemic attack (TIA).

In some embodiments, the human patient is screened for peripheral vascular disease (PVD). In some embodiments, the human patient does not have, or is selected for not having PVD.

In some embodiments, the human patient is screened for ischemic bowel disease. In some embodiments, the human patient does not have, or is selected for not having ischemic bowel disease.

In some embodiments, the human patient is screened for uncontrolled hypertension. In some embodiments, the human patient does not have, or is selected for not having uncontrolled hypertension.

In some embodiments, the human patient is screened to determine whether they are moderate to severe renal insufficiency patients who are at risk for renal failure due to volume depletion. In some embodiments, the human patient does not have, or is selected for not being moderate to severe renal insufficiency patients who are at risk for renal failure due to volume depletion.

In some embodiments, the human patient is screened for recent use (i.e., within 24 hours) of an ergotamine-containing medication, or ergot-type medication (such as dihydroergotamine or methysergide). In some embodiments, the human patient has not, or is selected for not recently used (i.e., within 24 hours) of an ergotamine-containing medication, or ergot-type medication (such as dihydroergotamine or methysergide)

In some embodiments, the human patient is screened for concurrent administration or recent discontinuation (i.e., within 2 weeks) of a MAO-A inhibitor. In some embodiments, the human patient does not have, or is selected for not having concurrent administration or recent discontinuation (i.e., within 2 weeks) of a MAO-A inhibitor.

In some embodiments, the human patient is screened for hemiplegic or basilar migraine. In some embodiments, the human patient does not have, or is selected for not having

In some embodiments, the human patient is monitored for life-threatening disturbances of cardiac rhythm, including ventricular tachycardia and ventricular fibrillation leading to death., and the subject dosage form is discontinued if these disturbances occur.

As with other 5-HT1 agonists, sensations of tightness, pain, pressure, and heaviness in the precordium, throat, neck and jaw commonly occur after treatment with the subject dosage form and are usually non-cardiac in origin. However, in some embodiments, a cardiac origin is suspected, the human patient is evaluated. In some embodiments, the evaluation reveals that the human patient does not have CAD or Prinzmetal's variant angina, and treatment with the subject dosage form is continued.

Cerebral hemorrhage, subarachnoid hemorrhage, and stroke have occurred in patients treated with 5-HT1 agonists, and some have resulted in fatalities. In a number of cases, it appears possible that the cerebrovascular events were primary, the 5-HT1 agonist having been administered in the incorrect belief that the symptoms experienced were a consequence of migraine, when they were not.

In some embodiments, the human patient is monitored for a cerebrovascular event (e.g., stroke, hemorrhage, transient ischemic attack). If no cerebrovascular event (e.g., stroke, hemorrhage, transient ischemic attack) occurs, treatment with a subject dosage form is continued. If a cerebrovascular event occurs, treatment with the subject dosage form is discontinued.

In some embodiments, the human patient has not previously been diagnosed as a migraineur, and the human patient is evaluated to exclude other potentially serious neurological conditions before the subject dosage form is administered as described herein. In some embodiments, the human patient is migraineur who presents with atypical symptoms, and the human patient is evaluated to exclude other potentially serious neurological conditions before the subject dosage form is administered as described herein. In some embodiments, the human patient is evaluated to exclude a history of stroke or transient ischemic attack before the subject dosage form is administered as described herein. 5-HT1 agonists, including the subject dosage form, may cause non-coronary vasospastic reactions, such as peripheral vascular ischemia, gastrointestinal vascular ischemia and infarction (presenting with abdominal pain and bloody diarrhea), splenic infarction, and Raynaud's syndrome. In some embodiments, the human patient has experienced symptoms or signs suggestive of non-coronary vasospasm reaction following the use of a 5-HT1 agonist, and the suspected vasospasm reaction is ruled out before receiving the subject dosage form, or before receiving additional doses of the subject dosage for.

NSAIDs, including meloxicam, can lead to new onset of hypertension or worsening of pre-existing hypertension, either of which may contribute to the increased incidence of CV events. Patients taking angiotensin converting enzyme (ACE) inhibitors, thiazides or loop diuretics may have impaired response to these therapies when taking NSAIDs [see Drug Interactions (7.1)].

Significant elevation in blood pressure (BP), including hypertensive crisis with acute impairment of organ systems, has been reported on rare occasions in patients with and without a history of hypertension receiving 5-HT1 agonists, including rizatriptan. In healthy young adult male and female patients who received maximal doses of rizatriptan (10 mg every 2 hours for 3 doses), slight increases in BP (approximately 2-3 mmHg) were observed. In some embodiments, the human patient is monitored or evaluated for uncontrolled hypertension. In some embodiments, the human patient does not have, or is selected for not having, uncontrolled hypertension.

In some embodiments, the blood pressure of the human patient is monitored during the initiation of the subject dosage form treatment. In some embodiments, the blood pressure of the human patient is monitored throughout the course of therapy. In some embodiments, the blood pressure of the human patient is monitored during the initiation of the subject dosage form treatment and throughout the course of therapy.

In some embodiments, the lowest effective dosage of the subject dosage form is administered the shortest possible duration.

In some embodiments, the subject dosage form is not administered with another NSAID.

In some embodiments, the subject dosage form is administered to the human patient for treatment of migraine, and the human patient and those responsible for the care of the human patient are advised to remain alert for signs and symptoms of GI ulceration and bleeding.

In some embodiments, the human patient is suspected to have a serious GI adverse event, the human patient is evaluated and treated for a serious GI adverse event, and the subject dosage form is not administered again until a serious GI adverse event is ruled out.

In some embodiments, the subject dosage form is administered to a human patient who is concomitantly using aspirin (such as low-dose aspirin) for cardiac prophylaxis, and the human patient is monitored for evidence of GI bleeding.

In some embodiments, the human patient is informed of the warning signs and symptoms of hepatotoxicity (e.g., nausea, fatigue, lethargy, diarrhea, pruritus, jaundice, right upper quadrant tenderness, and “flu-like” symptoms), and treatment is continued only as long as clinical signs and symptoms consistent with liver disease do not develop, and if systemic manifestations do not occur (e.g., eosinophilia, rash, etc.).

In some embodiments, the human patient treated with the subject dosage form has a sign or symptom of anemia, and the hemoglobin or hematocrit of the patient is monitored.

In some embodiments, the human patient has a co-morbid condition such as a coagulation disorders, or is concomitantly using of warfarin, other anticoagulants, antiplatelet agents (e.g., aspirin), SSRIs, an SNRIs, or a combination thereof, and the human patient is monitored for signs of bleeding.

The pharmacological activity of the subject dosage form in reducing inflammation, and possibly fever, may diminish the utility of diagnostic signs in detecting infections.

In some embodiments, the human patient being treated with the subject dosage form is periodically monitored with a complete blood count (CBC) and/or a chemistry profile.

In some embodiments, the human patient being is treated with the subject dosage and concomitant use of anticoagulants (e.g., warfarin), antiplatelet agents (e.g., aspirin), SSRIs, and SNRIs is monitored for signs of bleeding.

In some embodiments, the human patient being treated with the subject dosage form is not receiving concomitant analgesic doses of aspirin.

In some embodiments, the human patient being treated with the subject dosage form is receiving concomitant low-dose aspirin for cardiac prophylaxis, and the human patient is monitored more closely for evidence of GI bleeding.

In some embodiments, the human patient being treated with the subject dosage form, and there is no indication that the human patient is experiencing serotonin syndrome.

In some embodiments, the human patient is concomitantly treated with the subject dosage form and an ACE-inhibitor, an ARB, or a beta blocker, and the human patient's blood pressure is monitored. In some embodiments, the human patient is hydrated. In some embodiments, the renal function of the human patient is assessed at the beginning of the concomitant treatment and periodically thereafter.

In some embodiments, the human patient is concomitantly treated with the subject dosage form an ACE-inhibitors or an ARBs, wherein the human patient is elderly, volume-depleted, or has impaired renal function, and the human patient is monitored for signs of worsening renal function. In some embodiments, the human patient is hydrated. In some embodiments, the renal function of the human patient is assessed at the beginning of the concomitant treatment and periodically thereafter.

In some embodiments, the human patient is concomitantly treated with the subject dosage form and a diuretic, and the human patient is observed for signs of worsening renal function.

In some embodiments, the human patient is concomitantly treated with the subject dosage form and lithium, and the human patient is monitored for signs of lithium toxicity.

In some embodiments, the human patient is concomitantly treated with the subject dosage form and methotrexate, and the human patient is monitored for methotrexate toxicity.

In some embodiments, the human patient is concomitantly treated with the subject dosage form and cyclosporine, and the human patient is monitored for signs of worsening renal function.

In some embodiments, the human patient is treated with the subject dosage form, and the human patient does not receive another NSAID or a salicylate concomitantly.

In some embodiments, the human patient is concomitantly treated with the subject dosage form and pemetrexed, and the human patient has renal impairment and the human patient's creatinine clearance ranges from 45 to 79 mL/min, and the human patient is monitored for myelosuppression, renal and GI toxicity.

Patients taking the subject dosage form should interrupt dosing for at least five days before, the day of, and two days following pemetrexed administration.

In some embodiments, the human patient being treated with the subject dosage form is not undergoing treatment with a CYP2C9 inhibitor.

In some embodiments, the human patient being treated with the subject dosage form is not undergoing treatment with an ergotamine-containing or ergot-type medication (like dihydroergotamine or methysergide). In some embodiments, the subject dosage form is not administered within 24 hours of an ergotamine-containing or ergot-type medication (like dihydroergotamine or methysergide).

In some embodiments, the subject dosage form is not used within 24 hours of another 5HT1 agonist.

In some embodiments, the human patient being treated with the subject dosage form is not undergoing treatment with a concomitant MAO-A or a concomitant non-selective MAO inhibitor.

In some embodiments, wherein the human patient is a geriatric patient (age of 65 years or over) who has other cardiovascular risk factors (e.g., diabetes, hypertension, smoking, obesity, strong family history of coronary artery disease), the human patient has a cardiovascular evaluation prior to receiving the subject dosage form.

In some embodiments, the human patient who is being treated using the subject dosage form does not have severe renal impairment and is not at risk for renal failure due to volume depletion or for patients on hemodialysis.

In some embodiments, the human patient who is being treated using the subject dosage form does not have moderate to severe renal insufficiency and is not at risk for renal failure due to volume depletion.

In some embodiments, the human patient who is being treated using the subject dosage form has mild to moderate renal impairment.

The term “treating” or “treatment” includes the diagnosis, cure, mitigation, treatment, or prevention of disease in man or other animals, or any activity that otherwise affects the structure or any function of the body of man or other animals.

Patients who may benefit from the treatments described herein include pediatric patients, such as patients under about 18 years of age, about 0-5 years of age, about 5-10 years of age, about 10-12 years of age, or about 12-18 years of age; adult patients, such as patients having an age of about 18-65 years, about 18-30 years, about 30-50 years, about 50-65 years; and elderly patients, such as patients 65 years of age and over, about 65-75 years of age, about 75-90 years of age, or over 90 years of age.

In some embodiments, the human patient who is being treated using the subject dosage form has mild to moderate hepatic impairment.

The subject dosage form is a combination of rizatriptan and meloxicam formulated to improve the dissolution and absorption of meloxicam. After oral administration of a single dose of the subject dosage form under fasted conditions, meloxicam and rizatriptan are rapidly absorbed. The median Tₘₐₓ for meloxicam given as the subject dosage form is 0.88 hour which is different compared to standard oral meloxicam (Tₘₐₓ of 4-5 hours), and the median Tmax for rizatriptan given as the subject dosage form is 0.75 hour. Meloxicam when given in the subject dosage form has a mean Cₘₐₓ of approximately 2,900 ng/mL and a mean AUC0-24 of approximately 33,000 ng*hr/mL. Rizatriptan when given in the subject dosage form has a mean Cₘₐₓ of 32 ng/mL and a mean AUC0-24 of 83 ng*hr/mL.

The exposures of meloxicam and rizatriptan were comparable after administration of the subject dosage form in the fasted and fed states. Administration of the subject dosage form after a high-fat, high-calorie meal decreased the exposures of meloxicam by approximately 7% for AUC0-24 and 27% for Cₘₐₓ and increased the AUC0-24 of rizatriptan by approximately 10% with no significant change in Cₘₐₓ. Food intake delayed the time to maximal plasma concentration for both meloxicam (from 0.875 hours to 5 hours) and rizatriptan (from 0.75 hours to 1.5 hours).

Therapeutic concentrations of meloxicam were achieved within 1.5 hours with co-administration of food.

In some embodiments, the human patient who is being treated using the subject dosage form has mild renal impairment.

In some embodiments, the human patient who is being treated using the subject dosage form does not have moderate to severe renal impairment.

In some embodiments, the human patient who is being treated using the subject dosage form does not have moderate to severe renal insufficiency and is not at risk for renal failure due to volume depletion.

### 1 Indications and Usage

SYMBRAVO is indicated for the acute treatment of migraine with or without aura in adults.

### Limitations of Use

SYMBRAVO should only be used where a clear diagnosis of migraine has been established. If a patient has no response for the first migraine attack treated with SYMBRAVO, the diagnosis of migraine should be reconsidered before SYMBRAVO is administered to treat any subsequent attacks.
SYMBRAVO is not indicated for the preventive treatment of migraine attacks.
SYMBRAVO is not indicated for the treatment of cluster headache.

### 2 Dosage and Administration

### 2.1 Recommended Dose

The recommended dose of SYMBRAVO is one tablet (containing 20 mg meloxicam and 10 mg rizatriptan) by mouth, as needed for the acute treatment of migraine. The maximum daily dose should not exceed one tablet. The safety and effectiveness of a second dose for the same migraine attack have not been established. The safety of treating, on average, more than 7 headaches in a 30-day period has not been established.

Use for the shortest duration consistent with individual patient treatment goals [see Warnings and Precautions (5)].

### 2.2 Administration

Swallow SYMBRAVO tablets whole. Do not crush, divide, or chew the tablets. SYMBRAVO can be taken with or without food.

2.3 not Substitutable with Other Formulations of Meloxicam and of Rizatriptan

SYMBRAVO tablets have not shown equivalent systemic exposures to other formulations of oral meloxicam and of oral rizatriptan. Therefore, SYMBRAVO tablets are not substitutable with other formulations of oral meloxicam or oral rizatriptan products, even if the milligram strengths are the same. Do not substitute SYMBRAVO with similar dose strengths of other meloxicam or rizatriptan products [see Clinical Pharmacology (12.3)].

### 3 Dosage Forms and Strengths

Tablets: 20 mg meloxicam and 10 mg rizatriptan, white and modified capsule-shaped, debossed with “MXRZ” on one side and “20/10” on the other.

### 4 Contraindications

SYMBRAVO is contraindicated in patients with:
Ischemic coronary artery disease (angina pectoris, history of myocardial infarction, or documented silent ischemia), or other significant underlying cardiovascular disease [see Warnings & Precautions (5.1)].
Coronary artery vasospasm including Prinzmetal's angina [see Warnings & Precautions (5.1)].
In the setting of coronary artery bypass graft (CABG) surgery [see Warnings & Precautions (5.1)].
History of stroke or transient ischemic attack (TIA) [see Warnings & Precautions (5.4)].
Hemiplegic or basilar migraine.
Peripheral vascular disease (PVD) [see Warnings & Precautions (5.7)].
Ischemic bowel disease [see Warnings & Precautions (5.7)].
Uncontrolled hypertension [see Warnings & Precautions (5.9)].
Concomitant use of propranolol [see Drug Interactions (7.1)]
Recent use (i.e., within 24 hours) of an ergotamine-containing medication, ergot-type medication (such as dihydroergotamine or methysergide), or another 5-HT1 agonist (e.g., another triptan) [see Drug Interactions (7.1)].
Concurrent administration or recent discontinuation (i.e., within 2 weeks) of a MAO-A inhibitor [see Drug Interactions (7.1), Clinical Pharmacology (12.3)].
Known hypersensitivity (e.g., anaphylactic reactions and angioedema seen) to SYMBRAVO, meloxicam, rizatriptan, NSAIDs or any of the excipients in SYMBRAVO [see Warnings & Precautions (5.5), Adverse Reactions (6.2)].
History of asthma, urticaria, or other allergic-type reactions after taking aspirin or other NSAIDs. Severe, sometimes fatal anaphylactic-like reactions to NSAIDs have been reported in such patients [see Warnings & Precautions (5.16)].
Moderate to severe renal insufficiency in patients who are at risk for renal failure due to volume depletion or who are on dialysis [see Warnings & Precautions (5.11)].

### 5 Warnings and Precautions

### 5.1 Cardiovascular Thrombotic Events and Myocardial Infarction

Cardiovascular Thrombotic Events with NSAIDS

Clinical trials of several COX-2 selective and nonselective NSAIDs of up to three years duration have shown an increased risk of serious cardiovascular (CV) thrombotic events, including myocardial infarction (MI) and stroke, which can be fatal. Based on available data, it is unclear that the risk for CV thrombotic events is similar for all NSAIDs. The relative increase in serious CV thrombotic events over baseline conferred by NSAID use appears to be similar in those with and without known CV disease or risk factors for CV disease. However, patients with known CV disease or risk factors had a higher absolute incidence of excess serious CV thrombotic events, due to their increased baseline rate. Some observational studies found that this increased risk of serious CV thrombotic events began as early as the first weeks of treatment. The increase in CV thrombotic risk has been observed most consistently at higher doses.

To minimize the potential risk for an adverse CV event in NSAID-treated patients, use the lowest effective dose for the shortest duration possible. Physicians and patients should remain alert for the development of such events, throughout the entire treatment course, even in the absence of previous CV symptoms. Patients should be informed about the symptoms of serious CV events and the steps to take if they occur.

There is no consistent evidence that concurrent use of aspirin mitigates the increased risk of serious CV thrombotic events associated with NSAID use. The concurrent use of aspirin and an NSAID, such as meloxicam, increases the risk of serious gastrointestinal (GI) events [see Warnings & Precautions (5.2)].

### Status Post Coronary Artery Bypass Graft (CABG) Surgery

Two large, controlled clinical trials of a COX-2 selective NSAID for the treatment of pain in the first 10-14 days following CABG surgery found an increased incidence of MI and stroke. NSAIDs are contraindicated in the setting of CABG [see Contraindications (4)].

### Post-MI Patients

Observational studies conducted in the Danish National Registry have demonstrated that patients treated with NSAIDs in the post-MI period were at increased risk of reinfarction, CV-related death, and all-cause mortality beginning in the first week of treatment. In this same cohort, the incidence of death in the first year post-MI was 20 per 100 person years in NSAID-treated patients compared to 12 per 100 person years in non-NSAID exposed patients. Although the absolute rate of death declined somewhat after the first year post-MI, the increased relative risk of death in NSAID users persisted over at least the next four years of follow-up.

Myocardial Ischemia, Myocardial Infarction, and Prinzmetal's Angina with Rizatriptan

SYMBRAVO should not be given to patients with ischemic or vasospastic coronary artery disease. There have been rare reports of serious cardiac adverse reactions, including acute myocardial infarction, occurring within a few hours following administration of rizatriptan. Some of these reactions occurred in patients without known coronary artery disease (CAD). 5-HT₁ agonists, including SYMBRAVO may cause coronary artery vasospasm (Prinzmetal's Angina), even in patients without a history of CAD [see Contraindications (4)].

Triptan-naïve patients who have multiple cardiovascular risk factors (e.g., increased age, diabetes, hypertension, smoking, obesity, strong family history of CAD) should have a cardiovascular evaluation prior to receiving SYMBRAVO. If there is evidence of CAD or coronary artery vasospasm, SYMBRAVO should not be administered [see Contraindications (4)]. For patients who have a negative cardiovascular evaluation, consideration should be given to administration of the first dose of SYMBRAVO in a medically-supervised setting and performing an electrocardiogram (ECG) immediately following SYMBRAVO administration. Periodic cardiovascular evaluation should be considered in intermittent long-term users of SYMBRAVO who have cardiovascular risk factors.

Avoid the use of SYMBRAVO in patients with a recent MI unless the benefits are expected to outweigh the risk of recurrent CV thrombotic events. If SYMBRAVO is used in patients with a recent MI, monitor patients for signs of cardiac ischemia.

### 5.2 Gastrointestinal Bleeding, Ulceration, and Perforation

NSAIDs, including meloxicam, a component of SYMBRAVO, can cause serious GI adverse events including inflammation, bleeding, ulceration, and perforation of the esophagus, stomach, small intestine, or large intestine, which can be fatal. These serious adverse events can occur at any time, with or without warning symptoms, in patients treated with meloxicam. Only 1 in 5 patients who develop a serious upper GI adverse event on NSAID therapy is symptomatic. Upper GI ulcers, gross bleeding, or perforation caused by NSAIDs occurred in approximately 1% of patients treated for 3-6 months, and in about 2-4% of patients treated for one year. However, even short-term NSAID therapy is not without risk.

### Risk Factors for GI Bleeding, Ulceration, and Perforation

Patients with a prior history of peptic ulcer disease and/or GI bleeding who used NSAIDs had a greater than 10-fold increased risk for developing a GI bleed compared to patients without these risk factors. Other factors that increase the risk of GI bleeding in patients treated with NSAIDs include longer duration of NSAID therapy; concomitant use of oral corticosteroids, aspirin, anticoagulants, or selective SSRIs; smoking; use of alcohol; older age; and poor general health status. Most postmarketing reports of fatal GI events occurred in elderly or debilitated patients. Additionally, patients with advanced liver disease and/or coagulopathy are at increased risk for GI bleeding.

### Strategies to Minimize the GI Risks in NSAID-Treated Patients:

Use the lowest effective dosage for the shortest possible duration.
Avoid administration of more than one NSAID at a time.
Avoid use in patients at higher risk unless benefits are expected to outweigh the increased risk of bleeding. For such patients, as well as those with active GI bleeding, consider alternate therapies other than NSAIDs.
Remain alert for signs and symptoms of GI ulceration and bleeding during NSAID therapy.
If a serious GI adverse event is suspected, promptly initiate evaluation and treatment, and discontinue SYMBRAVO until a serious GI adverse event is ruled out.
In the setting of concomitant use of low-dose aspirin for cardiac prophylaxis, monitor patients more closely for evidence of GI bleeding [see Drug Interactions (7.1)].

### 5.3 Arrhythmias

Life-threatening disturbances of cardiac rhythm, including ventricular tachycardia and ventricular fibrillation leading to death, have been reported within a few hours following the administration of 5-HT₁ agonists. Discontinue SYMBRAVO if these disturbances occur.

### 5.4 Cerebrovascular Events

Cerebral hemorrhage, subarachnoid hemorrhage, and stroke have occurred in patients treated with 5-HT₁ agonists, and some have resulted in fatalities. In a number of cases, it appears possible that the cerebrovascular events were primary, the 5-HT₁ agonist having been administered in the incorrect belief that the symptoms experienced were a consequence of migraine, when they were not. Also, patients with migraine may be at increased risk of certain cerebrovascular events (e.g., stroke, hemorrhage, transient ischemic attack). Discontinue SYMBRAVO if a cerebrovascular event occurs.

Before treating headaches in patients not previously diagnosed with migraine, and in patients with migraine who present with atypical symptoms, care should be taken to exclude other potentially serious neurological conditions. SYMBRAVO is contraindicated in patients with a history of stroke or transient ischemic attack [see Contraindications (4)].

### 5.5 Anaphylactic Reactions

SYMBRAVO can cause anaphylactic reactions. Meloxicam has been associated with anaphylactic reactions in patients with and without known hypersensitivity to meloxicam and in patients with aspirin-sensitive asthma. Hypersensitivity reactions, including angioedema and anaphylaxis, have also occurred in patients receiving rizatriptan. Seek emergency help if an anaphylactic reaction occurs [see Contraindications (4), Warnings and Precautions (5.16)].

5.6 Chest, Throat, Neck and/or Jaw Pain/Tightness/Pressure

Sensations of tightness, pain, pressure, and heaviness in the precordium, throat, neck and jaw commonly occur after treatment with SYMBRAVO and are usually non-cardiac in origin. However, if a cardiac origin is suspected, patients should be evaluated. SYMBRAVO is contraindicated in patients with ischemic coronary artery disease and those with Prinzmetal's variant angina [see Contraindications (4)].

### 5.7 Other Vasospasm Reactions

5-HT₁ agonists, including SYMBRAVO, may cause non-coronary vasospastic reactions, such as peripheral vascular ischemia, gastrointestinal vascular ischemia and infarction (presenting with abdominal pain and bloody diarrhea), splenic infarction, and Raynaud's syndrome. In patients who experience symptoms or signs suggestive of non-coronary vasospasm reaction following the use of any 5-HT₁ agonist, the suspected vasospasm reaction should be ruled out before receiving additional SYMBRAVO doses.

Reports of transient and permanent blindness and significant partial vision loss have been reported with the use of 5-HT₁ agonists. Since visual disorders may be part of a migraine attack, a causal relationship between these events and the use of 5-HT₁ agonists has not been clearly established.

### 5.8 Hepatotoxicity

Elevations of ALT or AST (3- or more times the upper limit of normal [ULN]) have been reported in approximately 1% of NSAID-treated patients in clinical trials. In addition, rare, sometimes fatal, cases of severe hepatic injury, including fulminant hepatitis, liver necrosis, and hepatic failure have been reported.

Elevations of ALT or AST (less than 3-times ULN) may occur in up to 15% of patients treated with NSAIDs including meloxicam, a component of SYMBRAVO.

Inform patients of the warning signs and symptoms of hepatotoxicity (e.g., nausea, fatigue, lethargy, diarrhea, pruritus, jaundice, right upper quadrant tenderness, and “flu-like” symptoms). If clinical signs and symptoms consistent with liver disease develop, or if systemic manifestations occur (e.g., eosinophilia, rash, etc.), discontinue SYMBRAVO immediately, and perform a clinical evaluation of the patient [see Use in Specific Populations (8.7), Clinical Pharmacology (12.3)].

### 5.9 Hypertension/Increase in Blood Pressure

NSAIDs, including meloxicam, a component of SYMBRAVO, can lead to new onset of hypertension or worsening of pre-existing hypertension, either of which may contribute to the increased incidence of CV events. Patients taking angiotensin converting enzyme (ACE) inhibitors, thiazides or loop diuretics may have impaired response to these therapies when taking NSAIDs [see Drug Interactions (7.1)].

Significant elevation in blood pressure (BP), including hypertensive crisis with acute impairment of organ systems, has been reported on rare occasions in patients with and without a history of hypertension receiving 5-HT₁ agonists, including rizatriptan, a component of SYMBRAVO. In healthy young adult male and female patients who received maximal doses of rizatriptan (10 mg every 2 hours for 3 doses), slight increases in BP (approximately 2-3 mmHg) were observed. SYMBRAVO is contraindicated in patients with uncontrolled hypertension [see Contraindications (4)].

Monitor BP during the initiation of SYMBRAVO treatment and throughout the course of therapy.

### 5.10 Heart Failure and Edema

The Coxib and traditional NSAID Trialists' Collaboration meta-analysis of randomized controlled trials demonstrated an approximately 2-fold increase in hospitalizations for heart failure in COX-2 selective-treated patients and nonselective NSAID-treated patients compared to placebo-treated patients. In a Danish National Registry study of patients with heart failure, NSAID use increased the risk of MI, hospitalization for heart failure, and death.

Additionally, fluid retention and edema have been observed in some patients treated with NSAIDs. Use of SYMBRAVO may blunt the CV effects of several therapeutic agents used to treat these medical conditions (e.g., diuretics, ACE inhibitors, or angiotensin receptor blockers [ARBs]) [see Drug Interactions (7.1)].

Avoid the use of SYMBRAVO in patients with severe heart failure unless the benefits are expected to outweigh the risk of worsening heart failure. If SYMBRAVO is used in patients with severe heart failure, monitor patients for signs of worsening heart failure.

### 5.11 Renal Toxicity and Hyperkalemia

### Renal Toxicity

Long-term administration of NSAIDs has resulted in renal papillary necrosis, renal insufficiency, acute renal failure, and other renal injury.

Renal toxicity has also been seen in patients in whom renal prostaglandins have a compensatory role in the maintenance of renal perfusion. In these patients, administration of an NSAID may cause a dose-dependent reduction in prostaglandin formation and, secondarily, in renal blood flow, which may precipitate overt renal decompensation. Patients at greatest risk of this reaction are those with impaired renal function, dehydration, hypovolemia, heart failure, liver dysfunction, those taking diuretics and ACE inhibitors or ARBs, and the elderly. Discontinuation of NSAID therapy is usually followed by recovery to the pretreatment state.

SYMBRAVO is not recommended in patients with moderate to severe renal insufficiency and is contraindicated in patients with moderate to severe renal insufficiency who are at risk for renal failure due to volume depletion.

No information is available from controlled clinical studies regarding the use of meloxicam in patients with advanced renal disease. The renal effects of SYMBRAVO may hasten the progression of renal dysfunction in patients with pre-existing renal disease.

Correct volume status in dehydrated or hypovolemic patients prior to initiating SYMBRAVO. Monitor renal function in patients with renal or hepatic impairment, heart failure, dehydration, or hypovolemia during use of SYMBRAVO [see Drug Interactions (7.1)]. Avoid the use of SYMBRAVO in patients with advanced renal disease unless the benefits are expected to outweigh the risk of worsening renal function. If SYMBRAVO is used in patients with advanced renal disease, monitor patients for signs of worsening renal function [see Clinical Pharmacology (12.3)].

### Hyperkalemia

Increases in serum potassium concentration, including hyperkalemia, have been reported with use of NSAIDs, even in some patients without renal impairment. In patients with normal renal function, these effects have been attributed to a hyporeninemic-hypoaldosteronism state.

### 5.12 Serious Skin Reactions

NSAIDs, including SYMBRAVO, can cause serious skin adverse reactions such as exfoliative dermatitis, Stevens-Johnson Syndrome (SJS), and toxic epidermal necrolysis (TEN), which can be fatal. NSAIDs can also cause fixed drug eruption (FDE). FDE may present as a more severe variant known as generalized bullous fixed drug eruption (GBFDE), which can be life-threatening. These serious events may occur without warning. Inform patients about the signs and symptoms of serious skin reactions, and to discontinue the use of SYMBRAVO at the first appearance of skin rash or any other sign of hypersensitivity. SYMBRAVO is contraindicated in patients with previous serious skin reactions to NSAIDs [see Contraindications (4)].

5.13 Drug Reaction with Eosinophilia and Systemic Symptoms (DRESS)

Drug Reaction with Eosinophilia and Systemic Symptoms (DRESS) has been reported in patients taking NSAIDs such as SYMBRAVO. Some of these events have been fatal or life-threatening. DRESS typically, although not exclusively, presents with fever, rash, lymphadenopathy, and/or facial swelling. Other clinical manifestations may include hepatitis, nephritis, hematological abnormalities, myocarditis, or myositis. Sometimes symptoms of DRESS may resemble an acute viral infection. Eosinophilia is often present. Because this disorder is variable in its presentation, other organ systems not noted here may be involved. It is important to note that early manifestations of hypersensitivity, such as fever or lymphadenopathy, may be present even though rash is not evident. If such signs or symptoms are present, discontinue SYMBRAVO and evaluate the patient immediately.

### 5.14 Fetal Toxicity

### Premature Closure of Fetal Ductus Arteriosus

Avoid use of NSAIDs, including SYMBRAVO, in pregnant women at about 30 weeks gestation and later. NSAIDs, including SYMBRAVO, increase the risk of premature closure of the fetal ductus arteriosus at approximately this gestational age.

### Oligohydramnios/Neonatal Renal Impairment

Use of NSAIDs, including SYMBRAVO, at about 20 weeks gestation or later in pregnancy may cause fetal renal dysfunction leading to oligohydramnios and, in some cases, neonatal renal impairment. These adverse outcomes are seen, on average, after days to weeks of treatment, although oligohydramnios has been infrequently reported as soon as 48 hours after NSAID initiation. Oligohydramnios is often, but not always, reversible with treatment discontinuation. Complications of prolonged oligohydramnios may, for example, include limb contractures and delayed lung maturation. In some postmarketing cases of impaired neonatal renal function, invasive procedures such as exchange transfusion or dialysis were required.

If NSAID treatment is necessary between about 20 weeks and 30 weeks gestation, limit SYMBRAVO use to the lowest effective dose and shortest duration possible.

Consider ultrasound monitoring of amniotic fluid if SYMBRAVO treatment extends beyond 48 hours. Discontinue SYMBRAVO if oligohydramnios occurs and follow up according to clinical practice [see Use in Specific Populations (8.1)].

### 5.15 Hematologic Toxicity

Anemia has occurred in NSAID-treated patients. This may be due to occult or gross blood loss, fluid retention, or an incompletely described effect on erythropoiesis. If a patient treated with SYMBRAVO has any signs or symptoms of anemia, monitor hemoglobin or hematocrit.

NSAIDs, including SYMBRAVO, may increase the risk of bleeding events. Co-morbid conditions such as coagulation disorders or concomitant use of warfarin, other anticoagulants, antiplatelet agents (e.g., aspirin), SSRIs and SNRIs may increase this risk. Monitor these patients for signs of bleeding [see Drug Interactions (7.1)].

### 5.16 Exacerbation of Asthma Related to Aspirin Sensitivity

A subpopulation of patients with asthma may have aspirin-sensitive asthma which may include chronic rhinosinusitis complicated by nasal polyps; severe, potentially fatal bronchospasm; and/or intolerance to aspirin and other NSAIDs. Because cross-reactivity between aspirin and other NSAIDs has been reported in such aspirin-sensitive patients, SYMBRAVO is contraindicated in patients with this form of aspirin sensitivity [see Contraindications (4)]. When SYMBRAVO is used in patients with preexisting asthma (without known aspirin sensitivity), monitor patients for changes in the signs and symptoms of asthma.

### 5.17 Medication Overuse Headache

Overuse of acute migraine drugs (e.g., ergotamine, triptans, opioids, or a combination of drugs for 10 or more days per month) may lead to exacerbation of headache (medication overuse headache). Medication overuse headache may present as migraine-like daily headaches, or as a marked increase in frequency of migraine attacks. Detoxification of patients, including withdrawal of the overused drugs, and treatment of withdrawal symptoms (which often includes a transient worsening of headache) may be necessary.

### 5.18 Serotonin Syndrome

Serotonin syndrome may occur with triptans, including SYMBRAVO particularly during co-administration with selective serotonin reuptake inhibitors (SSRIs), serotonin norepinephrine reuptake inhibitors (SNRIs), tricyclic antidepressants (TCAs), and MAO inhibitors (MAOIs). Serotonin syndrome symptoms may include mental status changes (e.g., agitation, hallucinations, coma), autonomic instability (e.g., tachycardia, labile blood pressure, hyperthermia), neuromuscular aberrations (e.g., hyperreflexia, incoordination) and/or gastrointestinal symptoms (e.g., nausea, vomiting, diarrhea). The onset of symptoms can occur within minutes to hours of receiving a new or a greater dose of a serotonergic medication. SYMBRAVO treatment should be discontinued if serotonin syndrome is suspected [see Drug Interactions (7.1), Patient Counseling Information (17)].

### 5.19 Masking of Inflammation and Fever

The pharmacological activity of SYMBRAVO in reducing inflammation, and possibly fever, may diminish the utility of diagnostic signs in detecting infections.

### 5.20 Laboratory Monitoring

Because serious GI bleeding, hepatotoxicity, and renal injury can occur without warning symptoms or signs, consider monitoring patients on long-term NSAID treatment with a complete blood count (CBC) and a chemistry profile periodically [see Warnings and Precautions (5.2, 5.8, 5.11]. Discontinue SYMBRAVO if GI bleeding occurs or if abnormal liver or renal tests persist or worsen.

### 6 Adverse Reactions

The following adverse reactions are discussed in more detail in other sections of the labeling:
Cardiovascular Thrombotic Events and Myocardial Infarction [see Warnings and Precautions (5.1)]
GI Bleeding, Ulceration, and Perforation [see Warnings and Precautions (5.2)]
Arrhythmias [see Warnings and Precautions (5.3)]
Cerebrovascular Events [see Warnings and Precautions (5.4)]
Anaphylactic Reactions [see Warnings and Precautions (5.5)]
Chest, Throat, Neck and/or Jaw Pain/Tightness/Pressure [see Warnings and Precautions (5.6)]
Other Vasospasm Reactions [see Warnings and Precautions (5.7)]
Hepatotoxicity [see Warnings and Precautions (5.8)]
Hypertension/Increase in Blood Pressure [see Warnings and Precautions (5.9)]
Heart Failure and Edema [see Warnings and Precautions (5.10)]
Renal Toxicity and Hyperkalemia [see Warnings and Precautions (5.11)]
Serious Skin Reactions [see Warnings and Precautions (5.12)]
Drug Reaction with Eosinophilia and Systemic Symptoms (DRESS) [see Warnings and Precautions (5.13)]
Fetal Toxicity [see Warnings and Precautions (5.14)]
Hematologic Toxicity [see Warnings and Precautions (5.15)]
Exacerbation of Asthma Related to Aspirin Sensitivity [see Warnings and Precautions (5.16)]
Medication Overuse Headache [see Warnings and Precautions (5.17)]
Serotonin Syndrome [see Warnings and Precautions (5.18)]

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in clinical practice.

In two randomized, double-blind, controlled trials in adults with migraine, a total of 581 patients received a single dose of SYMBRAVO after the onset of a migraine attack (Studies 1 and 2) [see Clinical Studies (14)]. The most common adverse reactions from these two trials after treatment with SYMBRAVO (incidence 1% and greater than placebo) are provided in Table 1.

TABLE 1
Incidence (≥1% and Greater than Placebo) of Adverse Reactions
after a Single Dose of SYMBRAVO in Adults (Study 1 and Study 2)

| | | Rizatriptan | Meloxicam | |
|---|---|---|---|---|
| | SYMBRAVO | 10 mg | 20 mg | Placebo |
| | N = 581^{a} | N = 434^{b} | N = 433^{b} | N = 361^{a} |
| | % | % | % | % |
| Somnolence | 2 | 2 | 2 | 1 |
| Dizziness | 2 | 2 | 1 | 1 |

^{a}Study 1 and Study 2 pooled
^{b}Data from Study 1 only; Study 2 did not include arms with each individual component.

Long-term safety was assessed in 706 patients dosing intermittently for up to 12 months in an open-label extension trial where patients treated at least 2 migraines per month with SYMBRAVO. Of these 706 patients, 496 patients were exposed to SYMBRAVO for at least 6 months, and 132 were exposed for at least 12 months, all of whom treated at least 2 migraine attacks per month, on average.

### 6.2 Postmarketing Experience

The following adverse reactions have been reported with the individual components of SYMBRAVO, meloxicam and rizatriptan, from postmarketing experience. Because these reactions are reported voluntarily from a population of unknown size, it is not always possible to reliably estimate their frequency or establish a causal relationship to drug exposure.

### Meloxicam

Blood and Lymphatic System Disorders: Agranulocytosis
Hepatobiliary Disorders: Jaundice; liver failure
Psychiatric Disorders: Alterations in mood (such as mood elevation)
Renal and Urinary Disorders: Acute urinary retention; interstitial nephritis
Reproductive System and Breast Disorders: Infertility female
Skin and Subcutaneous Tissue Disorders: Anaphylactic reactions including shock; erythema multiforme; exfoliative dermatitis; Stevens-Johnson syndrome; fixed drug eruption (FDE); toxic epidermal necrolysis [see Warnings and Precautions (5.12)]

### Rizatriptan

General: Allergic conditions including anaphylaxis/anaphylactic reaction, angioedema, wheezing, and toxic epidermal necrolysis [see Contraindications (4)]
Neurological/Psychiatric: Seizure
Special Senses: Dysgeusia

### 7 Drug Interactions

7.1 Drugs Having Clinically Important Interactions with SYMBRAVO

See Table 2 for clinically significant drug interactions with SYMBRAVO [see Clinical Pharmacology (12.3)].

TABLE 2
Clinically Important Drug Interactions with SYMBRAVO
Drugs That Interfere with Hemostasis

| | |
|---|---|
| Clinical | Meloxicam and anticoagulants such as warfarin have a synergistic effect on |
| Impact | bleeding. The concomitant use of meloxicam and anticoagulants have an |
| | increased risk of serious bleeding compared to the use of either drug alone. |
| | Serotonin release by platelets plays an important role in hemostasis. Case- |
| | control and cohort epidemiological studies showed that concomitant use of |
| | drugs that interfere with serotonin reuptake and an NSAID may potentiate |
| | the risk of bleeding more than an NSAID alone. |
| Intervention | Monitor patients with concomitant use of SYMBRAVO with anticoagulants (e.g., |
| | warfarin), antiplatelet agents (e.g., aspirin), selective serotonin reuptake |
| | inhibitors (SSRIs), and serotonin norepinephrine reuptake inhibitors (SNRIs) for |
| | signs of bleeding [see Warnings and Precautions (5.15)]. Caution should be used |
| | when administering SYMBRAVO with warfarin since patients on warfarin may |
| | experience changes in International Normalized Ratio (INR) and an increased |
| | risk of bleeding complications when a new medication is introduced. |

Aspirin

| | |
|---|---|
| Clinical | Controlled clinical studies showed that the concomitant use of NSAIDs and |
| Impact | analgesic doses of aspirin does not produce any greater therapeutic effect than |
| | the use of NSAIDs alone. In a clinical study, the concomitant use of an NSAID |
| | and aspirin was associated with a significantly increased incidence of GI adverse |
| | reactions as compared to use of the NSAID alone [see Warnings and Precautions |
| | (5.2)]. |
| Intervention | Concomitant use of SYMBRAVO and analgesic doses of aspirin is not |
| | generally recommended because of the increased risk of bleeding [see |
| | Warnings and Precautions (5.15)]. |
| | In the setting of concomitant use of low-dose aspirin for cardiac prophylaxis, |
| | monitor patients more closely for evidence of GI bleeding [see Warnings and |
| | Precautions (5.2)]. |
| | Meloxicam in SYMBRAVO is not a substitute for low dose aspirin for |
| | cardiovascular protection. |

SSRIs/SNRIs and Serotonin Syndrome

| | |
|---|---|
| Clinical | Cases of serotonin syndrome have been reported during co-administration of |
| Impact | triptans and SSRIs or SNRIs [see Warnings and Precautions (5.18)]. |
| Intervention | SYMBRAVO treatment should be discontinued if serotonin syndrome is |
| | suspected. |

ACE Inhibitors, Angiotensin Receptor Blockers, and Beta-Blockers

| | |
|---|---|
| Clinical | NSAIDs may diminish the antihypertensive effect of angiotensin converting |
| Impact | enzyme (ACE) inhibitors, angiotensin receptor blockers (ARBs), or beta |
| | blockers. |
| | In patients who are elderly, volume-depleted (including those on diuretic |
| | therapy), or have renal impairment, co-administration of an NSAID with ACE |
| | inhibitors or ARBs may result in deterioration of renal function, including |
| | possible acute renal failure. These effects are usually reversible. |
| | Propranolol has been shown to increase the plasma AUC of rizatriptan. |
| Intervention | During concomitant use of SYMBRAVO and ACE-inhibitors, ARBs, or beta |
| | blockers, monitor blood pressure to ensure that the desired blood pressure |
| | is obtained. |
| | During concomitant use of SYMBRAVO and ACE-inhibitors or ARBs in |
| | patients who are elderly, volume-depleted, or have impaired renal function, |
| | monitor for signs of worsening renal function [see Warnings and Precautions |
| | (5.11)]. |
| | When these drugs are administered concomitantly, patients should be |
| | adequately hydrated. Assess renal function at the beginning of the |
| | concomitant treatment and periodically thereafter. |
| | The concomitant use of SYMBRAVO with propranolol is contraindicated. |

Diuretics

| | |
|---|---|
| Clinical | Clinical studies, as well as postmarketing observations, showed that NSAIDs |
| Impact | reduced the natriuretic effect of loop diuretics (e.g., furosemide) and thiazide |
| | diuretics in some patients. This effect has been attributed to the NSAID |
| | inhibition of renal prostaglandin synthesis. However, studies with furosemide |
| | agents and meloxicam have not demonstrated a reduction in natriuretic effect. |
| | Furosemide single and multiple dose pharmacodynamics and pharmacokinetics |
| | are not affected by multiple doses of meloxicam. |
| Intervention | During concomitant use of SYMBRAVO with diuretics, observe patients for signs |
| | of worsening renal function, in addition to assuring diuretic efficacy including |
| | antihypertensive effects [see Warnings and Precautions (5.11)]. |

Lithium

| | |
|---|---|
| Clinical | NSAIDs have produced elevations in plasma lithium levels and reductions in |
| Impact | renal lithium clearance. This effect has been attributed to NSAID inhibition of |
| | renal prostaglandin synthesis. |
| Intervention | During concomitant use of SYMBRAVO and lithium, monitor patients for signs |
| | of lithium toxicity. |

Methotrexate

| | |
|---|---|
| Clinical | Concomitant use of NSAIDs and methotrexate may increase the risk for |
| Impact | methotrexate toxicity (e.g., neutropenia, thrombocytopenia, renal |
| | dysfunction). |
| Intervention | During concomitant use of SYMBRAVO and methotrexate, monitor patients for |
| | methotrexate toxicity. |

Cyclosporine

| | |
|---|---|
| Clinical | Concomitant use of meloxicam and cyclosporine may increase cyclosporine's |
| Impact | nephrotoxicity. |
| Intervention | During concomitant use of SYMBRAVO and cyclosporine, monitor patients for |
| | signs of worsening renal function. |

NSAIDs and Salicylates

| | |
|---|---|
| Clinical | Concomitant use of meloxicam with other NSAIDs or salicylates (e.g., diflunisal, |
| Impact | salsalate) increases the risk of GI toxicity, with little or no increase in efficacy |
| | [see Warnings and Precautions (5.2)]. |
| Intervention | The concomitant use of SYMBRAVO with other NSAIDs or salicylates is not |
| | recommended. |

Pemetrexed

| | |
|---|---|
| Clinical | Concomitant use of meloxicam and pemetrexed may increase the risk of |
| Impact | pemetrexed-associated myelosuppression, renal, and GI toxicity (see the |
| | pemetrexed prescribing information). |
| Intervention | During concomitant use of SYMBRAVO and pemetrexed, in patients with renal |
| | impairment whose creatinine clearance ranges from 45 to 79 mL/min, monitor |
| | for myelosuppression, renal and GI toxicity. |
| | Patients taking SYMBRAVO should interrupt dosing for at least five days before, |
| | the day of, and two days following pemetrexed administration. In patients with |
| | creatinine clearance below 45 mL/min, the concomitant administration of |
| | SYMBRAVO with pemetrexed is not recommended. |

CYP2C9 Inhibitors

| | |
|---|---|
| Clinical | In vitro studies indicate that CYP2C9 (cytochrome P450 metabolizing enzyme) |
| Impact | plays an important role in the metabolic pathway of meloxicam with a minor |
| | contribution of the CYP3A4 isozyme. Thus, concomitant usage of CYP2C9 |
| | inhibitors (e.g., amiodarone, fluconazole) may lead to abnormally high plasma |
| | levels of meloxicam due to reduced metabolic clearance [see Use in Specific |
| | Populations (8.8), Clinical Pharmacology (12.3, 12.5)]. |
| Intervention | Use of SYMBRAVO in patients undergoing treatment with CYP2C9 inhibitors is |
| | not recommended. |

Ergot-Containing Drugs

| | |
|---|---|
| Clinical | Ergot-containing drugs have been reported to cause prolonged vasospastic |
| Impact | reactions. |
| Intervention | Because these effects may be additive with rizatriptan, use of ergotamine- |
| | containing or ergot-type medications (e.g., dihydroergotamine or methysergide) |
| | and SYMBRAVO within 24 hours is contraindicated [see Contraindications (4)]. |

5-HT₁ Agonists

| | |
|---|---|
| Clinical | Vasospastic effects may be additive with co-administration within 24 hours of |
| Impact | another 5-HT₁ agonists. |
| Intervention | Use of SYMBRAVO within 24 hours of another 5HT₁ agonist is contraindicated |
| | [see Contraindications (4)]. |

Monoamine Oxidase Inhibitors

| | |
|---|---|
| Clinical | MAO-A inhibitors increase the systemic exposure of rizatriptan and its |
| Impact | metabolite. |
| Intervention | SYMBRAVO is contraindicated in patients taking MAO-A inhibitors and non- |
| | selective MAO inhibitors [see Contraindications (4)]. |

### 8 Use in Specific Populations

### 8.1 Pregnancy

SYMBRAVO has not been studied in pregnant women. However, there are data pertaining to the use of the individual components, meloxicam and rizatriptan during pregnancy. These data are described below.

### Risk Summary

In the U.S. general population, the estimated background risk of major birth defects and miscarriage in clinically recognized pregnancies is 2% to 4% and 15% to 20%, respectively. The reported rate of major birth defects among infants born to women with migraine range from 2.2% to 2.9% and the reported rate of miscarriage was 17%, which are similar to rates reported in women without migraine.

### Meloxicam

Use of NSAIDs, including SYMBRAVO, can cause premature closure of the fetal ductus arteriosus and fetal renal dysfunction leading to oligohydramnios and, in some cases, neonatal renal impairment. Because of these risks, limit dose and duration of SYMBRAVO use between about 20 and 30 weeks of gestation, and avoid SYMBRAVO use at about 30 weeks of gestation and later in pregnancy [see Clinical Considerations, Data].

### Premature Closure of Fetal Ductus Arteriosus

Use of NSAIDs, including SYMBRAVO, at about 30 weeks gestation or later in pregnancy increases the risk of premature closure of the fetal ductus arteriosus.

### Oligohydramnios/Neonatal Renal Impairment

Use of NSAIDs at about 20 weeks gestation or later in pregnancy has been associated with cases of fetal renal dysfunction leading to oligohydramnios, and in some cases, neonatal renal impairment.

Data from observational studies regarding other potential embryofetal risks of NSAID use in women in the first or second trimesters of pregnancy are inconclusive.

In animal reproduction studies, embryofetal death was observed in rats and rabbits treated during the period of organogenesis with meloxicam at oral doses equivalent to 0.5 and 4.9 times, respectively, the maximum recommended human dose (MRHD) of 20 mg of meloxicam, based on body surface area (mg/m²). Increased incidence of septal heart defects was observed in rabbits treated throughout embryogenesis with meloxicam at an oral dose equivalent to 59 times the MRHD of 20 mg of meloxicam on a mg/m² basis. In pre- and post-natal reproduction studies, there was an increased incidence of dystocia, delayed parturition, and decreased offspring survival at 0.06 times the MRHD of 20 mg of meloxicam on a mg/m² basis. No teratogenic effects were observed in rats and rabbits treated with meloxicam during organogenesis at an oral dose equivalent to 2 and 20 times, respectively, the MRHD of 20 mg of meloxicam on a mg/m² basis [see Data].

Based on animal data, prostaglandins have been shown to have an important role in endometrial vascular permeability, blastocyst implantation, and decidualization. In animal studies, administration of prostaglandin synthesis inhibitors, such as meloxicam, resulted in increased pre- and post-implantation loss. Prostaglandins also have been shown to have an important role in fetal kidney development. In published animal studies, prostaglandin synthesis inhibitors have been reported to impair kidney development when administered at clinically relevant doses.

### Rizatriptan

Available human data on the use of rizatriptan in pregnant women are not sufficient to draw conclusions about drug-associated risk for major birth defects and miscarriage.

In animal studies, developmental toxicity was observed following oral administration of rizatriptan during pregnancy (decreased fetal body weight in rats) or throughout pregnancy and lactation (increased mortality, decreased body weight, and neurobehavioral impairment in rat offspring) at doses greater than the MRHD of 10 mg rizatriptan on a mg/m² basis [see Animal Data].

### Clinical Considerations

Disease-Associated Maternal and/or Embryo/Fetal Risk

In women with migraine, there is an increased risk of adverse perinatal outcomes in the mother, including pre-eclampsia and gestational hypertension.

### Fetal/Neonatal Adverse Reactions

### Premature Closure of Fetal Ductus Arteriosus:

Avoid use of NSAIDs in women at about 30 weeks gestation and later in pregnancy, because NSAIDs, including SYMBRAVO, can cause premature closure of the fetal ductus arteriosus [see Data].

### Oligohydramnios/Neonatal Renal Impairment

If an NSAID is necessary at about 20 weeks gestation or later in pregnancy, limit the use to the lowest effective dose and shortest duration possible. If SYMBRAVO treatment extends beyond 48 hours, consider monitoring with ultrasound for oligohydramnios. If oligohydramnios occurs, discontinue SYMBRAVO and follow up according to clinical practice [see Data].

### Labor or Delivery

There are no studies on the effects of SYMBRAVO during labor or delivery. In animal studies, NSAIDs, including meloxicam, inhibit prostaglandin synthesis, cause delayed parturition, and increase the incidence of stillbirth.

### Data

### Human Data for Meloxicam

### Premature Closure of Fetal Ductus Arteriosus:

Published literature reports that the use of NSAIDs at about 30 weeks of gestation and later in pregnancy may cause premature closure of the fetal ductus arteriosus.

### Oligohydramnios/Neonatal Renal Impairment:

Published studies and postmarketing reports describe maternal NSAID use at about 20 weeks gestation or later in pregnancy associated with fetal renal dysfunction leading to oligohydramnios, and in some cases, neonatal renal impairment. These adverse outcomes are seen, on average, after days to weeks of treatment, although oligohydramnios has been infrequently reported as soon as 48 hours after NSAID initiation. In many cases, but not all, the decrease in amniotic fluid was transient and reversible with cessation of the drug. There have been a limited number of case reports of maternal NSAID use and neonatal renal dysfunction without oligohydramnios, some of which were irreversible. Some cases of neonatal renal dysfunction required treatment with invasive procedures, such as exchange transfusion or dialysis.

Methodological limitations of these postmarketing studies and reports include lack of a control group; limited information regarding dose, duration, and timing of drug exposure; and concomitant use of other medications. These limitations preclude establishing a reliable estimate of the risk of adverse fetal and neonatal outcomes with maternal NSAID use.

Because the published safety data on neonatal outcomes involved mostly preterm infants, the generalizability of certain reported risks to the full-term infant exposed to NSAIDs through maternal use is uncertain.

### Human Data for Rizatriptan

In a pregnancy registry for rizatriptan users, no pattern of congenital anomalies or other adverse birth outcomes was identified over the period of 1998 to 2018. However, the lack of identification of any pattern should be viewed with caution, as the number of prospective reports with outcome information was low and did not provide sufficient power to detect an increased risk of individual birth defects associated with the use of rizatriptan. Additionally, there was significant loss to follow-up in the prospective pregnancy reports, further complicating this assessment of an association between rizatriptan and any pattern of congenital anomalies or other adverse birth outcomes.

In a study using data from the Swedish Medical Birth Register, live births to women who reported using triptans or ergots during pregnancy were compared with those of women who did not. Of the 157 births with first-trimester exposure to rizatriptan, 7 infants were born with malformations (relative risk 1.01 [95% Cl: 0.40 to 2.08]). A study using linked data from the Medical Birth Registry of Norway to the Norwegian Prescription Database compared pregnancy outcomes in women who redeemed prescriptions for triptans during pregnancy, as well as a migraine disease comparison group who redeemed prescriptions for triptans before pregnancy only, compared with a population control group. Of the 310 women who redeemed prescriptions for rizatriptan during the first trimester, 10 had infants with major congenital malformations (OR 1.03 [95% Cl: 0.55 to 1.93]), while for the 271 women who redeemed prescriptions for rizatriptan before, but not during, pregnancy, 12 had infants with major congenital malformations (OR 1.48 [95% Cl: 0.83 to 2.64]), each compared with the population comparison group.

### Animal Data

Animal reproduction studies have not been conducted for SYMBRAVO.

### Meloxicam

Meloxicam was not teratogenic when administered to pregnant rats during fetal organogenesis at oral doses up to 4 mg/kg/day (2-fold greater than the MRHD of 20 mg of meloxicam on a mg/m² basis). Administration of meloxicam to pregnant rabbits throughout embryogenesis produced an increased incidence of septal defects of the heart at an oral dose of 60 mg/kg/day (59-fold greater than the MRHD of 20 mg of meloxicam on a mg/m² basis). The no effect level was 20 mg/kg/day (20-fold greater than the MRHD of 20 mg of meloxicam based on BSA conversion). In rats and rabbits, embryolethality occurred at oral meloxicam doses of 1 mg/kg/day and 5 mg/kg/day, respectively (0.5 and 4.9-fold greater, respectively, than the MRHD of 20 mg of meloxicam on a mg/m² basis) when administered throughout organogenesis.

Oral administration of meloxicam to pregnant rats during late gestation through lactation increased the incidence of dystocia, delayed parturition, and decreased offspring survival at meloxicam doses of 0.125 mg/kg/day or greater (0.06 times the MRHD of 20 mg of meloxicam based on BSA comparison).

### Rizatriptan

When rizatriptan (0, 2, 10, or 100 mg/kg/day) was administered orally to pregnant rats throughout organogenesis, a decrease in fetal body weight was observed at the highest doses tested. The no-effect dose for adverse effects on embryofetal development was 10 mg/kg/day (10 times the MRHD of 10 mg of rizatriptan on a mg/m² basis). When rizatriptan (0, 5, 10, or 50 mg/kg/day) was administered orally to pregnant rabbits throughout organogenesis, no adverse fetal effects were observed. The highest dose tested of 50 mg/kg/day was 97 times the MRHD of 10 mg of rizatriptan on a mg/m² basis. Placental transfer of drug to the fetus was demonstrated in both species.

Oral administration of rizatriptan (0, 2, 10, or 100 mg/kg/day) to female rats prior to and during mating and continuing throughout gestation and lactation resulted in reduced body weight in offspring from birth and throughout lactation at all but the lowest dose tested (2 mg/kg/day). The no-effect dose (2 mg/kg/day) for adverse effects on postnatal development was 2 times the MRHD of 10 mg rizatriptan on a mg/m² basis.

Oral administration of rizatriptan (0, 5, 100, or 250 mg/kg/day) throughout organogenesis and lactation resulted in neonatal mortality, reduced body weight (which persisted into adulthood), and impaired neurobehavioral function in offspring at all but the lowest dose tested. The no-effect dose for adverse effects on postnatal development (5 mg/kg/day) was 5 times the MRHD of 10 mg rizatriptan on a mg/m² basis.

### 8.2 Lactation

### Risk Summary

### Meloxicam

There are no human data available on whether meloxicam is present in human milk, or on the effects on breastfed infants, or on milk production.

### Rizatriptan

There are no adequate data on the presence of rizatriptan or any active metabolites in human milk, or on the effects of rizatriptan on the breastfed infant, or on milk production. Rizatriptan was excreted in rat milk with levels in milk approximately 6 times those in maternal plasma.

The developmental and health benefits of breastfeeding should be considered along with the mother's clinical need for SYMBRAVO and any potential adverse effects on the breastfed infant from the SYMBRAVO or from the underlying maternal condition.

### Data

### Animal Data for Meloxicam

Meloxicam was present in the milk of lactating rats at concentrations higher than those in plasma.

### Animal Data for Rizatriptan

Following oral administration of rizatriptan to lactating rats at a dose of 100 mg/kg/day, drug concentrations of rizatriptan in milk samples exceeded maternal plasma drug concentrations by approximately 6-fold.

### 8.3 Females and Males of Reproductive Potential

### Infertility

### Females

Based on the mechanism of action, the use of prostaglandin-mediated NSAIDs, including SYMBRAVO, may delay or prevent rupture of ovarian follicles, which has been associated with reversible infertility in some women. Published animal studies have shown that administration of prostaglandin synthesis inhibitors has the potential to disrupt prostaglandin-mediated follicular rupture required for ovulation. Small studies in women treated with NSAIDs have also shown a reversible delay in ovulation. Consider withdrawal of SYMBRAVO in women who have difficulties conceiving or who are undergoing investigation of infertility.

Males Meloxicam in SYMBRAVO may compromise fertility in males of reproductive potential. In a published study, oral administration of meloxicam to male rats for 35 days resulted in decreased sperm count and motility and histopathological evidence of testicular degeneration at 0.5 times the MRHD on a mg/m² basis [see Nonclinical Toxicology (13.1)]. It is not known if these effects on fertility are reversible. The clinical relevance of these findings is unknown.

### 8.4 Pediatric Use

Safety and effectiveness of SYMBRAVO in pediatric patients have not been established.

### 8.5 Geriatric Use

Clinical studies of SYMBRAVO did not include subjects aged 65 and over to determine whether they respond differently from younger subjects.

Elderly patients, compared to younger patients, are at greater risk for NSAID-associated serious cardiovascular, gastrointestinal, hepatic, and/or renal adverse reactions. If the anticipated benefit for the elderly patient outweighs these potential risks, treat for the fewest number of days per month, as needed, and monitor patients for adverse effects [see Warnings and Precautions (5.1, 5.2, 5.8, 5.11)].

Geriatric patients who have other cardiovascular risk factors (e.g., diabetes, hypertension, smoking, obesity, strong family history of coronary artery disease) should have a cardiovascular evaluation prior to receiving SYMBRAVO [see Warnings and Precautions (5.1, 5.9)].

### 8.6 Renal Impairment

The use of SYMBRAVO, because of the meloxicam component, is contraindicated for patients with moderate to severe renal impairment who are at risk for renal failure due to volume depletion or for patients on hemodialysis, and is not recommended in patients with moderate to severe renal impairment [see Contraindications (4), Warnings and Precautions (5.11), Clinical Pharmacology (12.3)].

SYMBRAVO has not been studied in patients with renal impairment. The individual components, meloxicam and rizatriptan, have been studied and the results are described below.

### Meloxicam

Pharmacokinetics of meloxicam in elderly subjects with mild renal impairment is similar to healthy young subjects. Patients with moderate or severe renal impairment have not been studied. Meloxicam is not dialyzable.

### Rizatriptan

In patients with renal impairment (creatinine clearance 10-60 mL/min/1.73 m²), the AUC_{0-inf} Of rizatriptan was not significantly different from that in subjects with normal renal function. In hemodialysis patients, (creatinine clearance <2 mL/min/1.73 m²), however, the AUC for rizatriptan was greater than that in patients with normal renal function. Patients with severe renal impairment have not been studied [see Clinical Pharmacology (12.3)].

### 8.7 Hepatic Impairment

Use SYMBRAVO, because of the meloxicam component, with caution in patients with hepatic impairment, and monitor for adverse reactions when used in patients with severe hepatic impairment.

SYMBRAVO has not been studied in patients with hepatic impairment. The individual components, meloxicam and rizatriptan, have been studied and the results are described below.

### Meloxicam

Following oral administration of meloxicam, patients with mild to moderate hepatic impairment had no marked differences in plasma concentrations. Patients with severe hepatic impairment have not been adequately studied. Meloxicam is significantly metabolized in the liver and hepatotoxicity may occur [see Warnings and Precautions (5.8), Clinical Pharmacology (12.3)].

### Rizatriptan

Following oral administration of rizatriptan in patients with hepatic impairment caused by mild to moderate alcoholic cirrhosis of the liver, plasma concentrations of rizatriptan were similar in patients with mild hepatic impairment and greater in patients with moderate hepatic impairment, compared to a control group of subjects with normal hepatic function [see Clinical Pharmacology (12.3)].

### 8.8 Poor Metabolizers of CYP2C9 Substrates

In patients who are known or suspected to be poor CYP2C9 metabolizers based on genotype or previous history/experience with other CYP2C9 substrates (e.g., warfarin and phenytoin), the use of SYMBRAVO is not recommended as they may have abnormally high plasma levels due to reduced metabolic clearance of meloxicam. If SYMBRAVO is used in patients who are poor CYP2C9 metabolizers, monitor for adverse reactions.

### Overdosage

No overdoses of SYMBRAVO were reported during clinical trials in adults. Evaluation and treatment of SYMBRAVO overdose is based on experience with the individual components, meloxicam and rizatriptan.

In case of an overdosage, discontinue SYMBRAVO and contact a regional poison control center at 1-800-222-1222.

### Overdose of Meloxicam

Symptoms following acute meloxicam overdoses have been typically limited to lethargy, drowsiness, nausea, vomiting, and epigastric pain, which have been generally reversible with supportive care. Gastrointestinal bleeding has occurred. Hypertension, acute renal failure, respiratory depression and coma have occurred, but were rare [see Warnings and Precautions (5.2, 5.9, 5.11, 5.15)].

Manage patients with symptomatic and supportive care following a meloxicam overdosage. There are no specific antidotes. Consider emesis and/or activated charcoal (60 to 100 grams in adults, 1 to 2 grams per kg of body weight in pediatric patients) and/or osmotic cathartic in symptomatic patients seen within four hours of ingestion or in patients with a large overdosage (5 to 10 times the recommended dosage). Forced diuresis, alkalinization of urine, hemodialysis, or hemoperfusion may be employed but are not likely to be useful due to high protein binding.

There is limited experience with meloxicam overdose. In four reported cases of meloxicam overdose, patients took 6- to 11-times the highest available oral dose of meloxicam tablets (15 mg); all recovered. Cholestyramine is known to accelerate the clearance of meloxicam. Accelerated removal of meloxicam by 4 g doses of cholestyramine given three times a day was demonstrated in a clinical trial. Administration of cholestyramine may be useful following an overdosage.

### Overdose of Rizatriptan

In a clinical pharmacology study in which 12 adult subjects received rizatriptan at total cumulative doses of 80 mg (given within four hours), two of the subjects experienced syncope, dizziness, bradycardia including third degree AV block, vomiting, and/or incontinence.

Based on the pharmacology of rizatriptan, hypertension or myocardial ischemia could occur after overdosage. Gastrointestinal decontamination, (i.e., gastric lavage followed by activated charcoal) should be considered in patients suspected of an overdose with rizatriptan. Clinical and electrocardiographic monitoring should be continued for at least 12 hours, even if clinical symptoms are not observed. The effects of hemo- or peritoneal dialysis on serum concentrations of rizatriptan are unknown.

### 11 Description

SYMBRAVO contains meloxicam, a nonsteroidal anti-inflammatory drug (NSAID), and rizatriptan (as rizatriptan benzoate), a selective 5-HT_{1B/1D} receptor agonist.

### Meloxicam

Meloxicam has the molecular formula C₁₄H₁₃N₃O₄S₂ and is chemically designated as 4-hydroxy-2-methyl-N-(5 methyl-2-thiazolyl)-2H-1,2-benzothiazine-3-carboxamide-1,1-dioxide. It has a molecular weight of 351.4 g/mole.

The structural formula is:

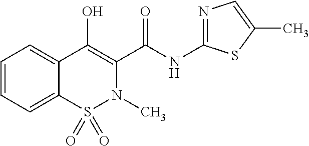 [SMILES: Cc1cnc(NC(=O)C2=C(O)c3ccccc3S(=O)(=O)N2C)s1]

Meloxicam is a pastel yellow solid, practically insoluble in water, with higher solubility observed in strong acids and bases. It is very slightly soluble in methanol. Meloxicam has an apparent partition coefficient (log P)=0.1 in n-octanol/buffer pH 7.4. Meloxicam has pKa values of 1.1 and 4.2.

### Rizatriptan

Rizatriptan benzoate has the molecular formula C₁₅H₁₉N₅·C₇H₆O₂ and is chemically designated as N,N-dimethyl-5-(1H-1,2,4-triazol-1-ylmethyl)-1H indole-3 ethanamine monobenzoate. The molecular weight of the free base rizatriptan is 269.4 g/mole. The structural formula is:

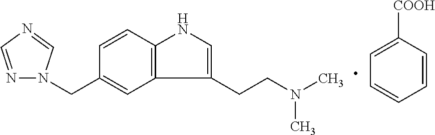 [SMILES: CN(C)CCc1c[nH]c2ccc(Cn3cncn3)cc12 ; O=C(O)c1ccccc1]

Rizatriptan benzoate is a white to off-white, crystalline solid that is soluble in water at about 42 mg per mL (expressed as free base) at 25° C.

Each SYMBRAVO tablet for oral administration contains 20 mg of meloxicam and 10 mg of rizatriptan (equivalent to 14.5 mg of rizatriptan benzoate). Each tablet also contains the excipients colloidal silicon dioxide, crospovidone, magnesium stearate, microcrystalline cellulose, partially hydrolyzed polyvinyl alcohol, polyethylene glycol, povidone, pregelatinized starch, sodium bicarbonate, sulfobutyl-ether-R-cyclodextrin sodium, talc, and titanium dioxide.

### 12 Clinical Pharmacology

### 12.1 Mechanism of Action

SYMBRAVO contains meloxicam and rizatriptan.

Meloxicam has analgesic, anti-inflammatory, and antipyretic properties. The mechanism of action of meloxicam, like that of other NSAIDs, is not completely understood but involves inhibition of cyclooxygenase (COX-1 and COX-2).

Meloxicam is a potent inhibitor of prostaglandin synthesis in vitro. Meloxicam concentrations reached during therapy have produced in vivo effects. Prostaglandins sensitize afferent nerves and potentiate the action of bradykinin in inducing pain in animal models. Prostaglandins are mediators of inflammation. Because meloxicam is an inhibitor of prostaglandin synthesis, its mode of action may be due to a decrease of prostaglandins in peripheral tissues.

Rizatriptan binds with high affinity to human cloned 5-HT_{1B/1D} receptors. Rizatriptan presumably exerts its therapeutic effects in the treatment of migraine headache by binding to 5-HT_{1B/1D} receptors located on intracranial blood vessels and sensory nerves of the trigeminal system.

### 12.2 Pharmacodynamics

### Cardiac Electrophysiology

### Rizatriptan

The effects of rizatriptan on QT prolongation have not been studied; however, arrhythmias have occurred after administration of 5-HT₁ agonists [see Warnings and Precautions (5.3)].

### Blood Pressure

### Rizatriptan

Significant elevation in blood pressure, including hypertensive crisis, has been reported in patients treated with rizatriptan, with and without a history of hypertension [see Warnings and Precautions (5.9)].

### 12.3 Pharmacokinetics

SYMBRAVO is a combination of rizatriptan and meloxicam. Meloxicam when given as SYMBRAVO has a mean Cₘₐₓ of approximately 2,900 ng/mL and a mean AUC₀₋₂₄ of approximately 33,000 ng*hr/mL. Rizatriptan when given as SYMBRAVO has a mean Cₘₐₓ of 32 ng/mL and a mean AUC₀₋₂₄ of 83 ng*hr/mL.

### Absorption

After oral administration of a single dose of SYMBRAVO under fasted conditions, the median Tₘₐₓ for the meloxicam component is 0.88 hours, which is less than oral meloxicam tablets (Tₘₐₓ of 4-hours). The median Tₘₐₓ for the rizatriptan component is 0.75 hours.

### Effect of Food

The exposures of meloxicam and rizatriptan were comparable after administration of SYMBRAVO in the fasted and fed states. Administration of SYMBRAVO after a high-fat, high-calorie meal decreased the exposures of meloxicam by approximately 7% for AUC₀₋₂₄ and 27% for Cₘₐₓ and increased the AUC₀₋₂₄ of rizatriptan by approximately 10% with no significant change in Cₘₐₓ. Food intake delayed the time to maximal plasma concentration for both meloxicam (from 0.88 hours to 5 hours) and rizatriptan (from 0.75 hours to 1.5 hours). Therapeutic concentrations of meloxicam were achieved within 1.5 hours with co-administration of food [see Dosage and Administration (2.2)].

### Distribution

### Meloxicam

The apparent volume of distribution during the terminal elimination phase (Vz) of meloxicam is approximately 10 L.

Meloxicam is approximately 99.4% bound to human plasma proteins (primarily albumin) within the therapeutic dose range. The fraction of protein binding is independent of drug concentration, over the clinically relevant concentration range, but decreases to approximately 99% in patients with renal disease. Meloxicam penetration into human red blood cells, after oral dosing, is less than 10%. Following a radiolabeled dose, over 90% of the radioactivity detected in the plasma was present as unchanged meloxicam.

### Rizatriptan

The mean volume of distribution of rizatriptan is approximately 140 liters in male subjects and 110 liters in female subjects. Rizatriptan is minimally bound (14%) to plasma proteins.

### Elimination

### Metabolism

### Meloxicam

Meloxicam is extensively metabolized in the liver. Meloxicam metabolites include 5′-carboxy meloxicam (60% of dose), from cytochrome P450 (CYP) mediated metabolism formed by oxidation of an intermediate metabolite, 5′-hydroxymethyl meloxicam, which is also excreted to a lesser extent (9% of dose). In vitro studies indicate that CYP2C9 plays an important role in this metabolic pathway with a minor contribution of the CYP3A4 isozyme. Patients' peroxidase activity is probably responsible for the other two metabolites, which account for 16% and 4% of the administered dose, respectively. The four metabolites are not known to have any in vivo pharmacological activity.

### Rizatriptan

The primary route of rizatriptan metabolism is via oxidative deamination by monoamine oxidase-A (MAO-A) to the indole acetic acid metabolite, which is not active at the 5-HT_{1B/1D} receptor. N-monodesmethyl-rizatriptan, a metabolite with activity similar to that of the parent compound at the 5-HT_{1B/1D} receptor, is formed to a minor degree. Plasma concentrations of N-monodesmethyl-rizatriptan are approximately 14% of those of parent compound, and it is eliminated at a similar rate. Other minor metabolites, the N-oxide, the 6-hydroxy compound, and the sulfate conjugate of the 6-hydroxy metabolite are not active at the 5-HT_{1B/1D} receptor.

After administration of SYMBRAVO, mean elimination half-life is approximately 18 hours for meloxicam and 2 hours for rizatriptan.

### Excretion

### Meloxicam

Meloxicam excretion is predominantly in the form of metabolites and occurs to equal extents in the urine and feces. Only traces of the unchanged parent compound are excreted in the urine (0.2%) and feces (1.6%). The extent of the urinary excretion was confirmed for unlabeled multiple 7.5 mg doses: 0.5%, 6%, and 13% of the dose were found in urine in the form of meloxicam, and the 5′-hydroxymethyl and 5′-carboxy metabolites, respectively. There is significant biliary and/or enteral secretion of the drug. This was demonstrated when oral administration of cholestyramine following a single intravenous dose of meloxicam decreased the AUC of meloxicam by 50%. The mean elimination half-life (t_{1/2}) ranges from 15 hours to 20 hours. Plasma clearance ranges from 7 to 9 mL/min.

### Rizatriptan

The total radioactivity of the administered dose recovered over 120 hours in urine and feces was 82% and 12%, respectively, following a single 10 mg oral administration of ¹⁴C-rizatriptan. Following oral administration of ¹⁴C-rizatriptan, rizatriptan accounted for about 17% of circulating plasma radioactivity. Approximately 14% of an oral dose is excreted in urine as unchanged rizatriptan while 51% is excreted as indole acetic acid metabolite, indicating substantial first pass metabolism. The plasma half-life of rizatriptan in males and females averages 2-3 hours.

### Cytochrome P450 Isoforms

Rizatriptan is not an inhibitor of the activities of human liver cytochrome P450 isoforms 3A4/5, 1A2, 2C9, 2C19, or 2E1; rizatriptan is a competitive inhibitor (Kᵢ=1400 nM) of cytochrome P450 2D6, but only at high, clinically irrelevant concentrations.

### Specific Populations

Studies have not been conducted to assess the pharmacokinetics of SYMBRAVO in geriatric patients or in patients with renal or hepatic impairment.

Based on population pharmacokinetics analyses, age and gender do not have a clinically meaningful effect on the pharmacokinetics of meloxicam.

### Geriatric Patients

Rizatriptan pharmacokinetics in healthy elderly volunteers without migraine (age 65-77 years) were similar to those in younger volunteers without migraine (age 18-45 years).

Male and Female Patients The mean AUC_{0-∞} and Cₘₐₓ of rizatriptan (10 mg orally) were about 30% and 11% higher in females as compared to males, respectively, while Tₘₐₓ occurred at approximately the same time.
Patients with Renal Impairment

### Meloxicam

The pharmacokinetics of meloxicam have been investigated in elderly subjects with mild renal impairment (eGFR 60-90) compared to young healthy volunteers. A 5% and 7% increase of Cₘₐₓ and AUC, respectively, was observed in elderly subjects with mild renal impairment. Use of meloxicam in patients with moderate or severe renal impairment have not been studied [see Use in Specific Populations (8.6)].

### Rizatriptan

In patients with renal impairment (creatinine clearance 10-60 mL/min/1.73 m²), the AUC_{0-∞} of rizatriptan was not significantly different from that in subjects with normal renal function. In hemodialysis patients, (creatinine clearance <2 mL/min/1.73 m²), however, the AUC for rizatriptan was approximately 44% greater than that in patients with normal renal function. Use of rizatriptan in patients with moderate and severe renal impairment have not been adequately studied.

Patients with Hepatic Impairment

### Meloxicam

Following a single 15 mg dose of meloxicam tablets there was no marked difference in plasma concentrations in patients with mild (Child-Pugh Class I) or moderate (Child-Pugh Class II) hepatic impairment compared to healthy volunteers. Protein binding of meloxicam was not affected by hepatic impairment. Use of meloxicam in patients with severe hepatic impairment (Child-Pugh Class III) have not been adequately studied [see Warnings and Precautions (5.8), Use in Specific Populations (8.7)].

### Rizatriptan

Following oral administration of rizatriptan in patients with hepatic impairment caused by mild to moderate alcoholic cirrhosis of the liver, plasma concentrations of rizatriptan were similar in patients with mild hepatic impairment compared to a control group of subjects with normal hepatic function; plasma concentrations of rizatriptan were approximately 30% greater in patients with moderate hepatic impairment.

### Drug Interaction Studies

### Aspirin:

When NSAIDs were administered with aspirin, the protein binding of NSAIDs were reduced, although the clearance of free NSAID was not altered. The clinical significance of this interaction is not known [see Drug Interactions (7.1)].

### Cholestyramine:

Pretreatment for four days with cholestyramine significantly increased the clearance of meloxicam by 50%. This resulted in a decrease in t_{1/2}, from 19.2 hours to 12.5 hours, and a 35% reduction in AUC. This suggests the existence of a recirculation pathway for meloxicam in the gastrointestinal tract. The clinical relevance of this interaction has not been established.

### Cimetidine:

Concomitant administration of 200 mg cimetidine four times daily did not alter the single-dose pharmacokinetics of 30 mg meloxicam.

### Digoxin:

Meloxicam tablets 15 mg once daily for 7 days did not alter the plasma concentration profile of digoxin after p-acetyldigoxin administration for 7 days at clinical doses. In vitro testing found no protein binding drug interaction between digoxin and meloxicam [see Drug Interactions (7.1)].

### Lithium:

In a study conducted in healthy subjects, mean pre-dose lithium concentration and AUC were increased by 21% in subjects receiving lithium doses ranging from 804 to 1072 mg twice daily with meloxicam tablets 15 mg once per day every day as compared to subjects receiving lithium alone [see Drug Interactions (7.1)].

### Methotrexate:

A study in 13 rheumatoid arthritis (RA) patients evaluated the effects of multiple oral doses of meloxicam on the pharmacokinetics of methotrexate taken once weekly. Meloxicam did not have a significant effect on the pharmacokinetics of single doses of methotrexate. In vitro, methotrexate did not displace meloxicam from its human serum binding sites [Drug Interactions (7.1)].

### Monoamine Oxidase Inhibitors:

Rizatriptan is principally metabolized via monoamine oxidase, ‘A’ subtype (MAO-A). Plasma concentrations of rizatriptan may be increased by drugs that are selective MAO-A inhibitors (e.g., moclobemide) or nonselective MAO inhibitors [type A and B] (e.g., isocarboxazid, phenelzine, tranylcypromine, and pargyline). In a drug interaction study, when 10 mg of rizatriptan was administered to subjects (n=12) receiving concomitant therapy with the selective, reversible MAO-A inhibitor, moclobemide 150 mg three times a day, there were mean increases in rizatriptan AUC and Cₘₐₓ of 119% and 41% respectively; and the AUC of the active N-monodesmethyl metabolite of rizatriptan was increased more than 400%. The interaction would be expected to be greater with irreversible MAO inhibitors. No pharmacokinetic interaction is anticipated in patients receiving selective MAO-B inhibitors [Contraindications (4)].

### Nadolol/Metoprolol:

In a drug interactions study, effects of multiple doses of nadolol 80 mg or metoprolol 100 mg every 12 hours on the pharmacokinetics of a single dose of 10 mg rizatriptan were evaluated in healthy subjects (n=12). No pharmacokinetic interactions were observed.

### Oral Contraceptives:

In a study of concurrent administration of an oral contraceptive during 6 days of administration of rizatriptan (10-30 mg/day) in healthy female volunteers (n=18), rizatriptan did not affect plasma concentrations of ethinyl estradiol or norethindrone.

### Propranolol:

In a study of concurrent administration of propranolol 240 mg/day and a single dose of rizatriptan 10 mg in healthy adult subjects (n=11), mean plasma AUC for rizatriptan was increased by 70% during propranolol administration, and a 4-fold increase was observed in one subject. The AUC of the active N-monodesmethyl metabolite of rizatriptan was not affected by propranolol [see Contraindications (4) and Drug Interactions (7.1)].

### Paroxetine:

In a study of the interaction between the SSRI paroxetine 20 mg/day for two weeks and a single dose of rizatriptan 10 mg in healthy subjects (n=12), neither the plasma concentrations of rizatriptan nor its safety profile were affected by paroxetine [see Warnings and Precautions (5.18), Drug Interactions (7.1), and Patient Counseling Information (17)].

### Warfarin:

The effect of meloxicam tablets on the anticoagulant effect of warfarin was studied in a group of healthy subjects receiving daily doses of warfarin that produced an International Normalized Ratio (INR) between 1.2 and 1.8. In these subjects, meloxicam did not alter warfarin pharmacokinetics and the average anticoagulant effect of warfarin as determined by prothrombin time. However, one subject showed an increase in INR from 1.5 to 2.1 [see Drug Interactions (7.1)].

### 12.5 Pharmacogenomics

CYP2C9 activity is reduced in individuals with genetic variants such as CYP2C9*2 and CYP2C9*3 polymorphisms. Limited data from three published reports showed that meloxicam AUC was substantially higher in individuals with reduced CYP2C9 activity, particularly in poor metabolizers (e.g., *3/*3), compared to normal metabolizers (*1/*1). The frequency of CYP2C9 poor metabolizer genotypes varies based on racial/ethnic background but is generally present in <5% of the population [see Use in Specific Populations (8.8)].

### 13 Nonclinical Toxicology

### 13.1 Carcinogenesis, Mutagenesis, Impairment of Fertility

The potential effects of the SYMBRAVO on carcinogenesis, mutagenesis, or impairment of fertility have not been studied. However, the individual components, meloxicam and rizatriptan, have been studied and the results of these studies are described below.

### Carcinogenesis

### Meloxicam

There was no increase in tumor incidence in long-term carcinogenicity studies in rats (104 weeks) and mice (99 weeks) administered meloxicam at oral doses up to 0.8 mg/kg/day in rats and up to 8.0 mg/kg/day in mice (up to 0.4 and 2 times, respectively, the MRHD of 20 mg/day of meloxicam on a mg/m² basis).

### Rizatriptan

Oral carcinogenicity studies of rizatriptan were conducted in mice (100 weeks) and rats (106 weeks) at doses of up to 125 mg/kg/day (up to 60 and 120 times, respectively, the MRHD of 10 mg of rizatriptan on a mg/m² basis). There was no evidence of an increase in tumor incidence related to rizatriptan in either species.

### Mutagenesis

### Meloxicam

Meloxicam was not mutagenic in an Ames assay. Meloxicam was not clastogenic in an in vitro chromosome aberration assay with human lymphocytes and an in vivo micronucleus assay in mouse bone marrow.

### Rizatriptan

Rizatriptan was neither mutagenic nor clastogenic in a battery of in vitro and in vivo genetic toxicity studies, including: the microbial mutagenesis (Ames) assay, in vitro mammalian cell mutagenesis and chromosomal aberration assays, and the in vivo chromosomal aberration assay in mouse.

### Impairment of Fertility

### Meloxicam

Meloxicam did not impair male and female fertility in rats at oral doses up to 9 mg/kg/day in males and 5 mg/kg/day in females (up to 4.4 and 2.4 times greater, respectively, than the MRHD of 20 mg of meloxicam on a mg/m² basis).

In a published study, oral administration of 1 mg/kg (0.5 times the MRHD on a mg/m² basis) meloxicam to male rats for 35 days resulted in decreased sperm count and motility and histopathological evidence of testicular degeneration. The clinical relevance of these findings is unknown.

### Rizatriptan

Oral administration of rizatriptan (0, 2, 10, or 100 mg/kg/day) to female rats prior to and during mating and continuing throughout gestation and lactation resulted in no effect on fertility; however, altered estrous cyclicity and delays in time to mating were observed at the highest dose tested. The no-effect dose for reproductive toxicity was approximately 10 mg/kg/day (10 times the MRHD of 10 mg rizatriptan on a mg/m² basis). Oral administration of rizatriptan (0, 5, 35, or 250 mg/kg/day) to male rats prior to and during mating resulted in no impairment of fertility or reproductive performance. The highest dose tested (250 mg/kg/day) was 242 times the MRHD of 10 mg rizatriptan on a mg/m² basis.

### 14 Clinical Studies

### Study 1: Efficacy Trial

The efficacy of SYMBRAVO for the acute treatment of migraine with or without aura in adults was demonstrated in one randomized, double-blind, controlled trial [Study 1 (NCT03896009)]. SYMBRAVO demonstrated an effect on headache pain freedom and most bothersome symptom (MBS) freedom at two hours after dosing, compared to placebo. Among patients who selected an MBS, the most commonly selected symptom was photophobia (61%), followed by nausea (20%), and phonophobia (19%).

In Study 1, 1,594 patients with a history of migraine with or without aura, according to the International Classification of Headache Disorders (ICHD-3) diagnostic criteria, were randomized to receive either SYMBRAVO (N=456), 10 mg rizatriptan (N=456), 20 mg meloxicam (matching the formulation of the meloxicam used in SYMBRAVO) (N=455), or placebo (N=227). Patients were instructed to treat a migraine of moderate to severe pain intensity with a single dose of medication. Rescue medication (including triptans and NSAIDs) was allowed 2 hours after the initial treatment; however, ergots were not allowed within 24 hours before or after study drug administration, and opioids were not allowed for the duration of the study. Patients were 83% female and 17% male, predominantly White (77%), with a mean age of 41.2 years (range 18-67). Approximately 7% of patients were taking preventive medications for migraine at baseline.

The primary efficacy analyses were conducted in patients who treated a migraine with moderate to severe pain. Pain freedom was defined as a reduction of moderate or severe headache pain to no headache pain, and MBS freedom was defined as the absence of the self-identified MBS (i.e., photophobia, phonophobia, or nausea). The percentage of patients achieving headache pain freedom and MBS freedom 2 hours after a single dose was statistically significantly greater among patients who received SYMBRAVO compared to those who received placebo (Table 3).

The key secondary endpoint of percentage of patients who experienced sustained pain freedom up to 24 hours (pain free from 2 hours through 24 hours postdose without use of other medications) was statistically significantly greater among patients who received a single dose of SYMBRAVO (16.1%) compared to those who received meloxicam (9%; p=0.001), or rizatriptan (11%; p=0.038).

Additional secondary endpoints included pain relief (reduction in migraine pain intensity from moderate or severe to mild or no headache pain) and the ability to perform normal daily activities at 2 hours after dosing. The ability to perform normal daily activities at two hours after dosing was derived from a single item questionnaire, which asked patients to select one response on a 4-point scale; normal function, mild impairment, severe impairment, or required bedrest. A statistically significantly greater percentage of participants who received SYMBRAVO achieved pain relief at 2 hours after dosing and were able to perform normal daily activities at 2 hours after dosing when compared to placebo (Table 3).

TABLE 3
Migraine Efficacy Endpoints for SYMBRAVO (Study 1)^{a}

| | | | |
|---|---|---|---|
| SYMBRAVO | Rizatriptan | Meloxicam | Placebo |
| (N = 428) | (N = 419) | (N = 421) | (N = 209) |

Co-Primary Endpoints

| | | | | |
|---|---|---|---|---|
| Pain Free at Hour 2 | 19.9%^{b} | 17.4% | 11.6% | 6.7% |
| MBS Free at Hour 2 | 36.9%^{b} | 35.8% | 32.5% | 24.4% |

Key-Secondary Endpoint

| | | | | |
|---|---|---|---|---|
| 24 Hour Sustained | 16.1%^{c} | 11.2% | 8.8% | 5.3% |
| Pain Freedom | | | | |

Other Secondary Endpoints

| | | | | |
|---|---|---|---|---|
| Pain Relief at Hour 2 | 69.2%^{b} | 65.6% | 62.0% | 54.5% |
| Able to Perform | 32.0%^{b} | 30.3% | 24.5% | 21.1% |
| Normal Daily | | | | |
| Activities at Hour 2 | | | | |

^{a}p-values provided only for prespecified comparisons.
^{b}p < 0.01 versus placebo.
^{c}p < 0.05 versus rizatriptan, and meloxicam.

Use of rescue medication within 24 hours was also evaluated. There was a numerically lower percentage of patients who received SYMBRAVO and used a rescue medication within 24 hours (23%) vs rizatriptan (35%), meloxicam (35%), and placebo (44%).

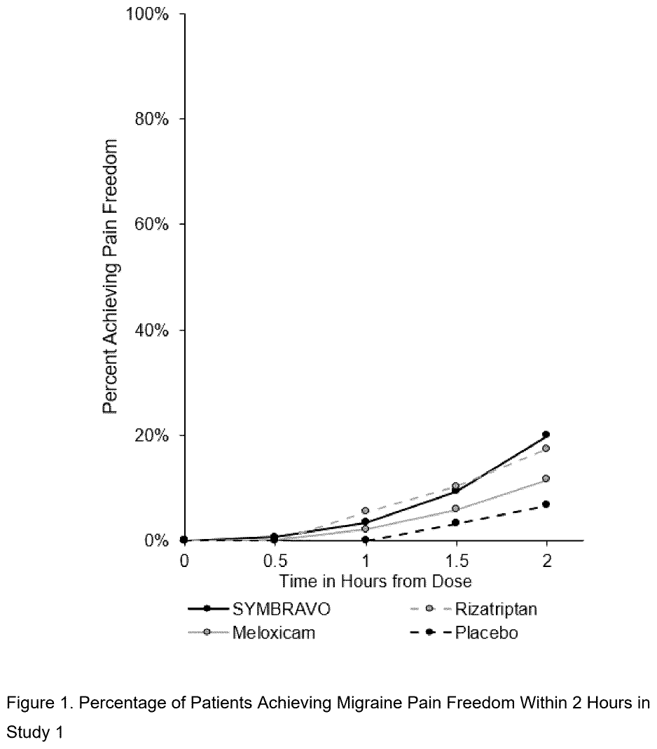

FIG. 1 presents the percentage of patients achieving migraine pain freedom within 2 hours following treatment in Study 1.

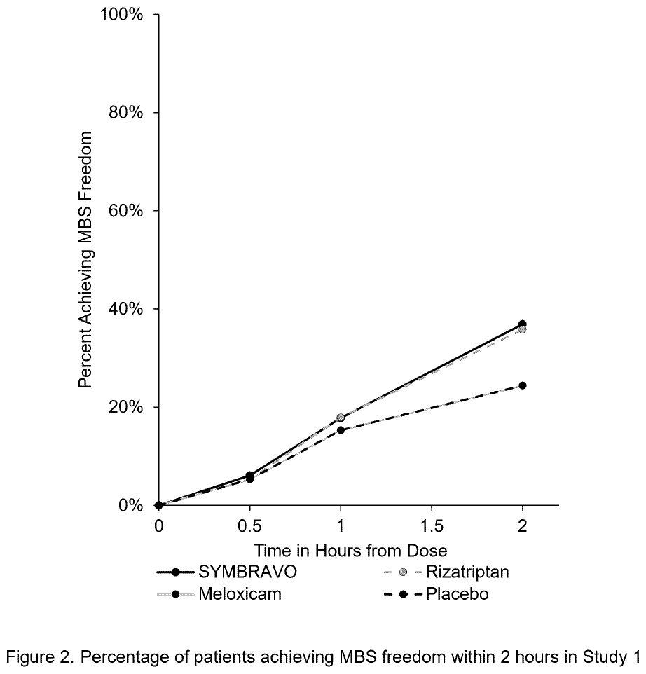

FIG. 2 presents the percentage of patients achieving MBS freedom within 2 hours following treatment Study 1.

The incidence of photophobia and phonophobia freedom at 2 hours was reduced following administration of SYMBRAVO as compared to placebo, with 33.5% and 23.5%. of subjects, respectively, reporting absence of photophobia, and 40.0% vs 18.6% of patients, respectively, reporting absence of phonophobia.

Study 2: Treatment of a Migraine when Mild in Severity

In Study 2 (NCT04163185), 302 patients with a history of migraine with or without aura, according to the ICHD-3 diagnostic criteria, were randomized to either SYMBRAVO (N=152) or placebo (N=150). Patients were instructed to treat a migraine when the initial pain was mild, with a single dose of medication. Rescue medication was allowed 2 hours after the initial treatment. Patients were 85.4% female and 14.6% male, predominantly White (83.1%), with a mean age of 41.5 years (range 19-65), and a mean BMI of 28.5 kg/m² (range 17.2-39.9). Approximately 6.4% of patients were taking preventive medications for migraine at baseline.

The primary efficacy analyses were conducted in patients who treated a migraine with initial pain that was mild. The percentage of patients achieving headache pain freedom and MBS freedom 2 hours after a single dose was statistically significantly greater among patients who received SYMBRAVO compared to those who received placebo (Table 4). Pain freedom at 2 hours was 32.6% in the SYMBRAVO treated group, compared to 16.3% in placebo (p=0.002). MBS freedom at 2 hours was 43.9% in the SYMBRAVO treated group, compared to 26.7% in the placebo group (p=0.003) (Table 4).

TABLE 4
Migraine Efficacy Endpoints for SYMBRAVO in Treatment
of a Migraine at Mild Pain Intensity (Study 2)

| | |
|---|---|
| SYMBRAVO | Placebo |

Pain Free at 2 hours

| | | |
|---|---|---|
| N | 132 | 135 |
| % Responder | 32.6% | 16.3% |
| p-value | 0.002 | |

Most Bothersome Symptom Free at 2 hours

| | | |
|---|---|---|
| N | 132 | 135 |
| % Responder | 43.9% | 26.7% |
| p-value | 0.003 | |

In Study 2, the percentage of patients with sustained pain freedom from 2 to 24 hours was numerically greater in SYMBRAVO than placebo (22.7% vs 12.6%) and rescue medication use within 24 hours was also numerically less in SYMBRAVO than placebo (15.2% vs 42.2%).

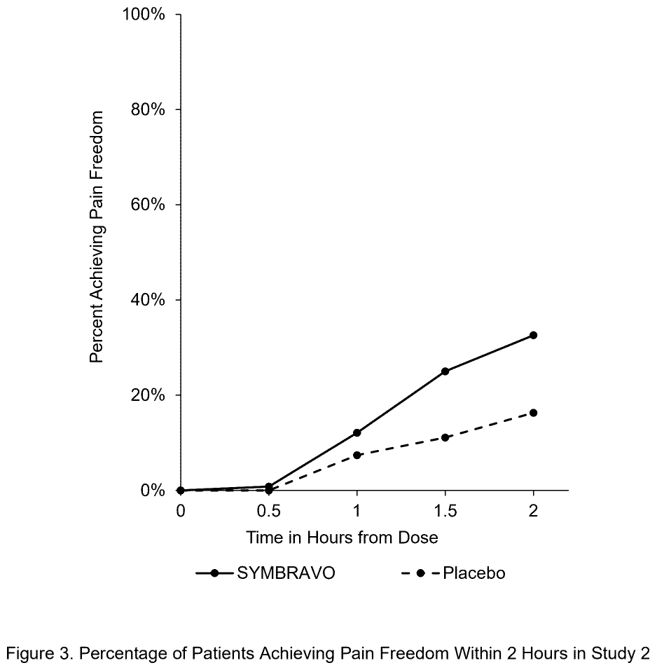

FIG. 3 presents the percentage of patients achieving migraine pain freedom within 2 hours following treatment in Study 2.

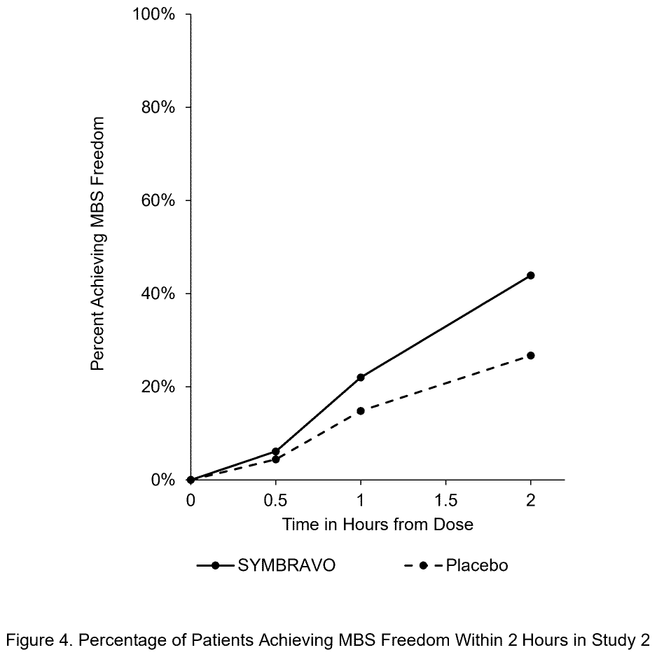

FIG. 4 presents the percentage of patients achieving MBS freedom within 2 hours following treatment Study 2.

The incidence of photophobia and phonophobia freedom at 2 hours was reduced following administration of SYMBRAVO as compared to placebo, with 43.2% vs 20.5%. of patients, respectively, reporting absence of photophobia, and 43.8% vs 25.0% of patients, respectively, reporting absence of phonophobia.

### 16 how Supplied/Storage and Handling

### 16.1 How Supplied

SYMBRAVO tablets are white, modified capsule-shaped, film-coated and debossed with “MXRZ” on one side and “20/10” on the other. SYMBRAVO is supplied in the following package configuration:

| Package | | |
|---|---|---|
| Configuration | Strength | NDC Code |
| Bottles of | meloxicam 20 mg and | 81968-020-09 |
| 9 tablets | rizatriptan 10 mg | |

### 16.2 Storage and Handling

Store SYMBRAVO in the original bottle at 20° C. to 25° C. (68° F. to 77° F.), excursions permitted to 15° C. to 30° C. (59° F. to 86° F.) [see USP Controlled Room Temperature].

### 17 Patient Counseling Information

Advise the patient to read the FDA-approved patient labeling (Medication Guide).

### Cardiovascular Thrombotic Effects

Advise patients to be alert for the symptoms of cardiovascular thrombotic events, including chest pain, shortness of breath, weakness, or slurring of speech, and to report any of these symptoms to their healthcare provider immediately [see Warnings and Precautions (5.1)].

Risk of Myocardial Ischemia and/or Infarction, Prinzmetal's Angina, Other Vasospasm-Related Events, and Cerebrovascular Events

Inform patients that rizatriptan, a component of SYMBRAVO, may cause serious cardiovascular side effects such as myocardial infarction or stroke. Although serious cardiovascular events can occur without warning symptoms, patients should be alert for the signs and symptoms of chest pain, shortness of breath, weakness, slurring of speech, and should ask for medical advice when observing any indicative sign or symptoms. Patients should be apprised of the importance of this follow-up [see Warnings and Precautions (5.1, 5.3, 5.4, 5.6, 5.7)].

### Gastrointestinal Bleeding, Ulceration, and Perforation

Advise patients to report symptoms of ulcerations and bleeding, including epigastric pain, dyspepsia, melena, and hematemesis to their healthcare provider. In the setting of concomitant use of low-dose aspirin for cardiac prophylaxis, inform patients of the increased risk for the signs and symptoms of GI bleeding [see Warnings and Precautions (5.2)].

### Anaphylactic Reactions

Inform patients of the signs of an anaphylactic reaction (e.g., difficulty breathing, swelling of the face or throat). Instruct patients to seek immediate emergency help if these occur [see Contraindications (4) and Warnings and Precautions (5.5, 5.16)].

### Hepatotoxicity

Inform patients of the warning signs and symptoms of hepatotoxicity (e.g., nausea, fatigue, lethargy, diarrhea, pruritus, jaundice, right upper quadrant tenderness, and “flu-like” symptoms).

If these occur, instruct patients to stop SYMBRAVO and seek immediate medical therapy [see Warnings and Precautions (5.8)].

### Heart Failure and Edema

Advise patients to be alert for the symptoms of congestive heart failure including shortness of breath, unexplained weight gain, or edema and to contact their healthcare provider if such symptoms occur [see Warnings and Precautions (5.10)].

Serious Skin Reactions, including DRESS

Advise patients to stop SYMBRAVO immediately if they develop any type of rash or fever and to contact their healthcare provider as soon as possible [see Contraindications (4), Warnings and Precautions (5.12, 5.13)].

### Female Fertility

Advise females of reproductive potential who desire pregnancy that meloxicam, a component of SYMBRAVO, may be associated with a reversible delay in ovulation [see Use in Specific Populations (8.3)].

### Lactation

Advise patients to notify their healthcare provider if they are breastfeeding or plan to breastfeed [see Use in Specific Populations (8.2)].

### Fetal Toxicity

Inform pregnant women to avoid use of SYMBRAVO and other NSAIDs starting at 30 weeks gestation because of the risk of the premature closing of the fetal ductus arteriosus. If treatment with SYMBRAVO is needed for a pregnant woman between about 20 to 30 weeks gestation, advise her that she may need to be monitored for oligohydramnios, if treatment continues for longer than 48 hours [see Warnings and Precautions (5.14), Use in Specific Populations (8.1)].

### Avoid Concomitant Use of NSAIDs

Inform patients that the concomitant use of SYMBRAVO with other NSAIDs or salicylates (e.g., diflunisal, salsalate) is not recommended due to the increased risk of gastrointestinal toxicity, and little or no increase in efficacy [see Warnings and Precautions (5.5), Drug Interactions (7.1)]. Alert patients that NSAIDs may be present in “over the counter” medications for treatment of colds, fever, or insomnia.

### Use of NSAIDs and Low-Dose Aspirin

Inform patients not to use low-dose aspirin concomitantly with SYMBRAVO until they talk to their healthcare provider [see Drug Interactions (7.1)].

### Medication Overuse Headache

Inform patients that use of acute migraine drugs for 10 or more days per month may lead to an exacerbation of headache, and encourage patients to record headache frequency and drug use (e.g., by keeping a headache diary) [see Warnings and Precautions (5.17)].

### Serotonin Syndrome

Patients should be cautioned about the risk of serotonin syndrome with the use of SYMBRAVO, particularly during combined use with SSRIs or SNRIs [see Warnings and Precautions (5.18), Drug Interactions (7.1), Clinical Pharmacology (12.3)].

### Ability to Perform Complex Tasks

Since migraines or treatment with SYMBRAVO may cause somnolence and dizziness, instruct patients to evaluate their ability to perform complex tasks during migraine attacks and after administration of SYMBRAVO.

The Following patents are Incorporated by Reference in their Entireties:

U.S. Pat. Nos. 9,821,075, 10,029,010, 10,058,614, 10,137,131, 10,195,278, 10,265,324, 10,363,312, 10,369,224, 10,369,225, 10,426,839, 10,463,736, 10,471,014, 10,471,069, 10,512,692, 10,512,693, 10,517,950, 10,532,101, 10,537,642, 10,561,664, 10,583,144, 10,653,777, 10,688,185, 10,695,429, 10,695,430, 10,702,535, 10,702,602, 10,722,583, 10,729,696, 10,729,697, 10,729,773, 10,758,617, 10,758,618, 10,780,165, 10,780,166, 10,799,588, 10,821,181, 10,821,182, 10,894,053, 10,905,693, 10,918,722, 10,933,136, 10,933,137, 10,940,153, 10,987,358, 11,013,805, 11,013,806, 11,020,483, and 11,045,549.

Unless otherwise indicated, all numbers expressing quantities of ingredients, properties such as amounts, percentage, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the embodiments and does not pose a limitation on the scope of any claim. No language in the specification should be construed as indicating any non-claimed element essential to the practice of the claims.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or to expedite prosecution. When any such inclusion or deletion occurs, the specification is deemed to contain the group as modified thus fulfilling the written description of all Markush groups if used in the appended claims.

Certain embodiments are described herein, including the best mode known to the inventors for carrying out the claimed embodiments. Of course, variations on these described embodiments will become apparent to those of ordinary skill in the art upon reading the foregoing description. The inventor expects skilled artisans to employ such variations as appropriate, and the inventors intend for the claimed embodiments to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of treating migraine, comprising administering a tablet containing about 20 mg of meloxicam, and about 14.5 mg of rizatriptan benzoate to a human patient for the acute treatment of migraine, wherein the human patient has a clear diagnosis of migraine, wherein the human patient does not have moderate to severe renal impairment, wherein the maximum daily dose does not exceed one tablet, and wherein no more than 7 migraines are treated in a 30-day period.

2. (canceled)

3. (canceled)

4. The method of claim 1, wherein the tablet further contains sulfobutyl-ether-3-cyclodextrin sodium.

5. The method of claim 1, wherein the tablet further contains sodium bicarbonate.

6. The method of claim 1, wherein the tablet further contains partially hydrolyzed polyvinyl alcohol.

7. A method of treating migraine, comprising administering a tablet containing about 20 mg of meloxicam, and about 14.5 mg of rizatriptan benzoate to a human patient for the acute treatment of migraine, wherein the human patient has a clear diagnosis of migraine, wherein the human patient has been screened for renal impairment, wherein the maximum daily dose does not exceed one tablet, and wherein no more than 7 migraines are treated in a 30-day period.

8. (canceled)

9. (canceled)

10. The method of claim 7, wherein the tablet further contains sulfobutyl-ether-p-cyclodextrin sodium.

11. The method of claim 7, wherein the tablet further contains sodium bicarbonate.

12. The method of claim 7, wherein the tablet further contains partially hydrolyzed polyvinyl alcohol.

13. A method of treating migraine, comprising administering a tablet containing about 20 mg of meloxicam, and about 14.5 mg of rizatriptan benzoate to a human patient for the acute treatment of migraine, wherein the human patient has a clear diagnosis of migraine, wherein the human patient does not have hepatic impairment, wherein the maximum daily dose does not exceed one tablet, and wherein no more than 7 migraines are treated in a 30-day period.

14. (canceled)

15. (canceled)

16. The method of claim 13, wherein the tablet further contains sulfobutyl-ether-β-cyclodextrin sodium.

17. The method of claim 13, wherein the tablet further contains sodium bicarbonate.

18. The method of claim 13, wherein the tablet further contains partially hydrolyzed polyvinyl alcohol.

19. A method of treating migraine, comprising administering a tablet containing about 20 mg of meloxicam, and about 14.5 mg of rizatriptan benzoate to a human patient for the acute treatment of migraine, wherein the human patient has a clear diagnosis of migraine, wherein the human patient has been screened for hepatic impairment, wherein the maximum daily dose does not exceed one tablet, and wherein no more than 7 migraines are treated in a 30-day period.

20. (canceled)

21. (canceled)

22. The method of claim 19, wherein the tablet further contains sulfobutyl-ether-β-cyclodextrin sodium.

23. The method of claim 19, wherein the tablet further contains sodium bicarbonate.

24. The method of claim 19, wherein the tablet further contains partially hydrolyzed polyvinyl alcohol.

25. The method of claim 1, wherein the patient is pain free 2 hours after administration of the tablet containing about 20 mg of meloxicam, and about 14.5 mg of rizatriptan benzoate.

26. The method of claim 1, wherein the patient is MBS free 2 hours after administration of the tablet containing about 20 mg of meloxicam, and about 14.5 mg of rizatriptan benzoate, wherein the MBS is photophobia, photophobia followed by nausea, or phonophobia.
